SEQUENCE LISTING (29 sequences)

SEQ ID NO: 1 - DNA, 20 bp, Artificial Sequence
  Synthetic Sequence
aatgatacggcgaccaccga

SEQ ID NO: 2 - DNA, 24 bp, Artificial Sequence
  Synthetic sequence
caagcagaagacggcatacgagat

SEQ ID NO: 3 - DNA, 23 bp, Artificial Sequence
  Synthetic sequence
acactctttccctacacgacgct

SEQ ID NO: 4 - DNA, 37 bp, Artificial Sequence
  Synthetic sequence
cggtctcggcattcctgctgaaccgctcttccgatct

SEQ ID NO: 5 - DNA, 58 bp, Artificial Sequence
  Synthetic sequence
aatgatacggcgaccaccgagatctacactctttccctacacgacgctcttccgatct

SEQ ID NO: 6 - DNA, 61 bp, Artificial Sequence
  Synthetic sequence
caagcagaagacggcatacgagatcggtctcggcattcctgctgaaccgctcttccgatct

SEQ ID NO: 7 - DNA, 20 bp, Artificial Sequence
  Synthetic sequence
gctggtgctgcagcttatta

SEQ ID NO: 8 - DNA, 20 bp, Artificial Sequence
  Synthetic sequence
agggtcaagtgcacagtcta

SEQ ID NO: 9 - DNA, 20 bp, Artificial Sequence
  Synthetic sequence
acctccgactataagctgac

SEQ ID NO: 10 - DNA, 20 bp, Artificial Sequence
  Synthetic sequence
gctggtgctgcagcttatta

SEQ ID NO: 11 - DNA, 20 bp, Artificial Sequence
  Synthetic sequence
agggtcaagtgcacagtcta

SEQ ID NO: 12 - DNA, 10 bp, Artificial Sequence
  Sudden acute respiratory syndrome coronavirus 2
tatcttcaac

SEQ ID NO: 13 - DNA, 10 bp, Artificial Sequence
  Synthetic sequence
atagaacaac

SEQ ID NO: 14 - DNA, 20 bp, Artificial Sequence
  Synthetic sequence
aatacttgagaatacttgag

SEQ ID NO: 15 - DNA, 25 bp, Artificial Sequence
  Influenza A
caagaccaatcytgtcacctctgac

SEQ ID NO: 16 - DNA, 23 bp, Artificial Sequence
  Influenza A sequence
gcattytggacaaavcgtctacg

SEQ ID NO: 17 - DNA, 10 bp, Artificial Sequence
  Influenza A sequence
aggggatttt

SEQ ID NO: 18 - DNA, 10 bp, Artificial Sequence
  Synthetic sequence
tccctttttt

SEQ ID NO: 19 - DNA, 20 bp, Artificial Sequence
  Synthetic sequence
aatacttgagaatacttgag

SEQ ID NO: 20 - DNA, 22 bp, Artificial Sequence
  Influenza B sequence
tcctcaaytcactcttcgagcg

SEQ ID NO: 21 - DNA, 21 bp, Artificial Sequence
  Synthetic sequence
cggtgctcttgaccaaattgg

SEQ ID NO: 22 - DNA, 10 bp, Artificial Sequence
  Influenza B sequence
atgaaggaca

SEQ ID NO: 23 - DNA, 10 bp, Artificial Sequence
  Synthetic sequence
tacttcgaca

SEQ ID NO: 24 - DNA, 20 bp, Artificial Sequence
  Synthetic sequence
tcggtggtcgccgtatcatt

SEQ ID NO: 25 - DNA, 20 bp, Artificial Sequence
  Synthic Sequence
caccacgaggtaaggccagt

SEQ ID NO: 26 - DNA, 21 bp, Artificial Sequence
  Synthetic Sequence
ttgctttcgtggtattcttgc

SEQ ID NO: 27 - DNA, 24 bp, Artificial Sequence
  Synthetic Sequence
agactcacgttaacaatattgcag

SEQ ID NO: 28 - DNA, 11 bp, Artificial Sequence
  Synthetic Sequence
acactagccat

SEQ ID NO: 29 - DNA, 11 bp, Artificial Sequence
  Synthetic Sequence
tgtgatgccat

(19) United States
(12) Patent Application Publication
(10) Pub. No.: US 2024/0271181 A1
(43) Pub. Date: Aug. 15, 2024

(54) METHODS FOR DETECTING HOMOGENOUS TARGETS IN A POPULATION WITH NEXT GENERATION SEQUENCING

(71) Applicant: THE GOVERNMENT OF THE UNITED STATES AS REPRESENTED BY THE SECRETARY OF THE ARMY, Fort Detrick, MD (US)

(72) Inventors: Jeffrey R. KUGELMAN, New Market, MD (US); Peter A. LARSON, Frederick, MD (US); Raina KUMAR, Ashburn, VA (US); Mariano SANCHEZ-LOCKHART, New Market, MD (US)

(21) Appl. No.: 18/266,692

(22) PCT Filed: Dec. 10, 2021
(86) PCT No.: PCT/US21/62871

§ 371 Date: Jun. 12, 2023

Related U.S. Application Data
(60) Provisional application No. 63/123,707, filed on Dec. 10, 2020.

(51) Int. Cl. C12Q 1/6806; C12Q 1/6876; C40B 50/08

(52) U.S. Cl. CPC C12Q 1/6806; C12Q 1/6876; C40B 50/08

(57) ABSTRACT

Provided herein are methods for next generation sequencing a target using universal adapter technologies and identifying and reducing error by distinguishing the signals from sequencing the target based on the location on the flow cell of the signals from sequencing the sample identifying region(s) of the adapters.

### STATEMENT REGARDING FEDERALLY SPONSORED RESEARCH

This invention was made with government support under CV_20_ID_013 awarded by the United States Army Medical Research and Development Command. The U.S. government has certain rights in the invention.

### SEQUENCE LISTING

The instant application contains a Sequence Listing, which has been submitted electronically in ASCII format and is hereby incorporated by reference in its entirety. Said ASCII copy, created on Dec. 8, 2021, is named “RIID_21_02_ST25.txt” and is 5,002 bytes in size.

### BACKGROUND

The coronavirus disease 2019 (COVID-19) has required testing for the sudden acute respiratory syndrome coronavirus-2 (SARS-COV-2), the virus causing the disease, in larger and larger samples of the population. Quantitative and semi-quantitative polymerase chain reaction (qPCR and semi-qPCR respectively) use 96 or 384 well plates, limiting each detection run lasting one hour, which includes downtime.

A higher-throughput method is required for detecting SARS-COV-2. Next generation sequencing technologies were designed to sequence whole genomes in a few hours. In those next generation sequencing technologies that sequence-by-synthesis, a whole genome is fragmented into segments 50-300 base pairs in length; adapters are placed on the end of each fragment; and clusters are generated on a flow cell, each cluster being an amplification of one adapted fragment. Clusters are distinguished from one another by the electromagnetic signals (e.g., fluorescent signals) obtained from each cluster. Each electromagnetic signal for a cluster is the result of sequencing-by-synthesis wherein, a new single strand of the fragment is synthesized using the adapted single-stranded fragment as a template. When each nucleotide is added to the new single strand, an electromagnetic signal is released, and the order of the electromagnetic signals for a cluster corresponds to the order of nucleotides in the fragment. Each cluster has a different electromagnetic signal because generally, fragments of genomic material are diverse. Thereby, each cluster is distinguished from the next by the differences in the electromagnetic signals obtained by sequencing the fragments. MISEQ® platforms can perform 45 million reads; NEXTSEQ500® 400 million; HISEQ RAPID® 600 million; NEXTSEQ2000® 1 billion; HISEQ® 2 billion; and NOVASEQ® 10 billion. Accordingly, with each new generation of sequencer, more reads of more discrete nucleotides provides for either the read of a larger genome or more samples.

In multiplexed sequencing, the genome of two or more subjects can be sequenced at the same time. Each pair of adapters has at least one index. The pair of adapters might have two, one on each adapter. Individually the index, or in combination the indices, contains a unique sequence distinguishing the genetic material of one individual from that of another. Even if the adapters have other unique sequences that identify a specific fragment (i.e., a barcode), all of the adapters for that individual will have the same unique index or indices. Barcodes may be used to identify errors in sequencing occurring in the process or may be used to determine polymorphisms within a single genome (i.e., cells having one sequence and another population of cells having another sequence, such as with a chimera). The sequencing of the indices or barcodes are performed after the first read sequencing of the target. The indices are around 6 to 8 nucleotides, and barcodes and indices may be up to 10 nucleotides in length.

In summary, the sequence of the target is used to distinguish one cluster or one signal from the next in the existing next generation sequencing methods using two indices. The sequence of the indices are read after at least the first read of the target and are used to distinguish from which individual the fragments in the cluster originated. FIG. 1.

### SUMMARY OF THE DISCLOSURE

The embodiments herein are based on the discovery that the existing methods of using conventional dual index adapter systems for next generation sequencing of a large sample population to detect SARS-COV-2 results in significant data loss.

In a dual indices system, 27% of samples containing SARS-COV-2 nucleic acids cannot be distinguished from signal noise when there are 96 samples. FIG. 6. It was considered that this high error rate was due to index swaps; when in fact, it was due to the inability of the next generation sequencing machine, algorithms, and software to distinguish one cluster from the next because such systems rely on difference in the target sequence to distinguish clusters and because the SARS-COV-2 nucleic acid was homogenous. With data correction, it can be reduced to 18%. FIG. 7. 27% or 18% of samples would need to be rerun, and as the number of samples are increased in the run, this percentage would increase. This data loss is high. In 5 out of 6 runs using the present methods with two indices that identifies SARS-COV-2 nucleic acids in 1000 samples, insufficient reads passing quality filters were detected to perform an analysis of SARS-CoV-2 positivity.

The significant data loss when detecting a homogeneous target appears in part to be due to the existing method's use of the sequence of the target to distinguish one cluster from another and the assumption that the sequences of the target fragments were diverse, as with genomic information. When the targets are homogenous, data loss occurs because one cluster cannot be distinguished from the next. While some embodiments detect SARS-COV-2, it should be understood that the other embodiments are not limited to detecting SARS-COV-2, and those embodiments detect other homogenous targets within a population. It should be understood that the present invention is not limited to the detection of the targets described herein, but includes detection of any homogenous target with at least a partially known sequence wherein target primers can be generated capturing a length of the target that can be sequenced-by-synthesis on the next generation sequencing machines.

Some embodiments herein are based on the solution that a single sample identifying region may be doubled in length (e.g., at least 16 nucleotides) compared to the length of indices or barcodes, and the sequence of the sample identifying region may be: 1) read before, 2) in-line, or 3) before and in-line with that of the target, thereby providing the ability to distinguish clusters by the sequence of the sample identifying region or to distinguish the signals obtained by sequencing the target in different samples by the location on the flow cell of the signals obtained from sequencing the subject identifying regions. These solutions unexpectedly provide for less loss of samples or a reduced need to rerun samples because the initial sequencing was not able to resolve identifying signals for these samples.

### BRIEF DESCRIPTION OF THE DRAWINGS

The detailed description of preferred embodiments of the invention will be better understood when read in conjunction with the appended drawings. For the purpose of illustrating the invention, the drawings show certain, but not all, preferred embodiments. It should be understood that embodiments of the invention are not limited to the precise arrangements and instrumentalities of those shown in the drawings.

FIG. 1 depicts the error profile of next generation sequencing using two adapters, each having an index, and detecting 96 samples. As each nucleotide is read in the target, the next generation sequencer should be able to resolve the signals from individual clusters (shown from having faint, broad bands to having narrow, intense bands) as more nucleotide sequence information is obtained for each target. However, with detection of a homogenous target, i.e., SARS-COV-2 nucleic acid, it takes more reads of nucleotides in the sequence (i.e., cycles on the next generation sequencer) to be able to resolve the signals from individual clusters, if at all. When increased to 1000 samples, in five out of six experiments, the next generation sequencer was not able to resolve individual clusters. As the number of samples increases with existing methods, there is more information to resolve and more difficulty distinguishing individual clusters.

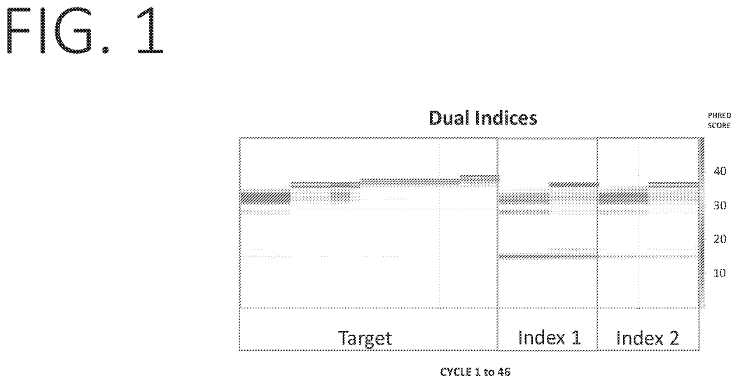

FIG. 2 depicts the structure of single-strand ribonucleic acid (RNA) for SARS-COV-2. In an exemplary embodiment, the target nucleic acid is within the sequence for the spike protein.

FIG. 3A depicts general strategies surrounding development and implementation of adapters, how they align with a target in the single-strand RNA of SARS-COV-2, and how the adapters can be elongated in first strand and second strand synthesis to obtain a second single-stranded product comprising the first primer binding region, first subject identifying region (SIR), the target, and the second primer binding region. In this illustration, the first primer binding region comprises a first sequencing primer binding region (i.e., a first read primer binding region) and a first substrate recognition region which can anneal to the first single-stranded nucleic acid bound to the substrate, and the second primer binding region comprising the second read primer binding region and the second substrate recognition region which can anneal to the second single-stranded nucleic acid bound to the substrate. However, in some embodiments the first single-stranded nucleic acid and the first read primer can bind to the same region, in part or wholly, in the first primer binding region, and in some embodiments the second single-stranded nucleic acid and the second read primer can bind to the same region, in part or wholly, in the second primer binding region. SEQ ID NOs: 1 & 2 are examples of the first and second single-stranded nucleic acid primer binding regions, respectively. SEQ ID NOs: 3 & 4 are examples of the first and second sequencing (read) primer binding regions, respectively. SEQ ID NOs: 5 & 6 are examples of the first and second primer binding regions. SEQ ID NOs: 7 and 8 are examples of the first and second target binding regions for SARS-COV-2.

FIG. 3B depicts reverse transcribing a single-stranded RNA to obtain cDNA.

FIG. 3C depicts annealing one of the first or second adapters to the cDNA that contains the target to produce a first strand comprising the target and either i) the second primer binding region or ii) the SIR and the first primer binding region, thereby obtaining a first single-stranded product. This illustration depicts the first adapter binding for simplicity.

FIG. 3D depicts annealing the other of the first or second adapters to the first single-stranded product thereby obtaining a second single-stranded product comprising the second primer binding region, the target, the SIR, and the first primer binding region. Subsequent amplification using the first and second adapters as “primers” is also depicted as a preferred embodiment.

FIG. 3E depicts the first portion of another method of producing the second single-stranded product wherein either a first primer comprising the first or second target primer is used for first single-stranded cDNA synthesis or the first single-stranded cDNA is produced by random primers to produce single-stranded cDNA. The first primer is then annealed and elongated to produce a first single-stranded cDNA.

FIG. 3F depicts the second portion of the other method of producing the second single-stranded product wherein a second primer comprising either the first or the second target primer is annealed to the first single-stranded cDNA to amplify the target thereby producing a first double-stranded cDNA. Then, two adapters are ligated to the first double-stranded DNA. Each adapter is ligated to the opposite end of the first double-stranded DNA from the other adapter. One adapter will comprise the first primer binding region and the SIR and the other adapter will comprise the second primer binding region. The adapter will anneal with the correct orientation (i.e., the SIR being proximal to the target and the first primer binding region being distal, and the second primer binding region also having the correct orientation). This orientation can be achieved by several methods. In this illustrative embodiment, a portion of each adapter is double-stranded (i.e., by comprising an intervening double-stranded region or by comprising a portion or all of the SIR or a portion of the second primer binding region that is annealed to the full length adapter portion, thereby creating a 5′ overhang for each adapter. The double-stranded portion will permit ligation with the appropriate orientation. The overhang can be undone by subsequently clongating the shorter strand. Ligation with the appropriate orientation can also be achieved using other methods, such as those involving a one nucleotide overhang, wherein the double-stranded cDNA has a nucleotide removed from one strand of each end. In the alternative, the adapters could be “Y” adapters, wherein the same adapter is ligated to each end. The “Y” adapter comprises a double-stranded intermediary sequence. One arm of the “Y” comprises the first primer binding region and the SIR, and the other arm of the “Y” comprises the second primer binding region. Unbound first and second single-stranded nucleic acids are then used to prime the first and second primer binding regions, thereby amplifying the sequences, thereby obtaining a product comprising, in order, the first primer binding region, the SIR, the target, and the second primer binding region.

FIG. 4 depicts a dual barcode adapter system, wherein the two barcodes are read separately from the target due to separate priming and depicts a single barcode adapter system, using unified primers for sequencing. The dual barcode adapter system requires custom primers for sequencing each target as well as primers for sequencing the barcode.

FIG. 5 depicts a comparison of a dual barcode design wherein the target is read first to an exemplary design, wherein the signals from reading the SIR are used to resolve the signals for the target. In this embodiment, the SIR is read before the target and in-line with the target.

FIG. 6 depicts the criteria, yields, error rate, projected throughput, heat maps including loss of samples, and normalization curves from studies comparing a dual barcode design, wherein the target is read first, to an exemplary design, wherein the signals from reading the SIR are used to resolve the signals for the target from a 96 sample study. The dual barcodes each are 10 nucleotides in length, and the SIR is 20 nucleotides in length. In this embodiment, the SIR is read before the target and in-line with the target. The heatmap demonstrates an unexpected result that the signals from the SIR design are unexpectedly more intense than the signals from the dual barcode design and that the results are more linear with the SIR design than with the dual barcode design. As a result, out of all of the samples known to carry the target, only in 4% was the presence of the target undetermined with the SIR design. This is unexpected compared to the dual barcode design, in which the same measure was 27%. Accordingly, with 96 sample run, the projected throughput is unexpectedly higher with the SIR design.

FIG. 7 depicts the same results as in FIG. 6 but with an informatics correction. Even with the corrections, the results with the SIR are unexpected.

FIG. 8 depicts the typical workflow and analysis of signals from the flow cell using a dual barcode adapter design; the dual barcodes each being 10 nucleotides in length. SARS-COV-2 nucleic acid was detected, and PhiX was used as a positive control. This was the analysis used to generate the results for the dual barcode in FIGS. 6 and 7.

FIG. 9 depicts an exemplary workflow and analysis of signals from the flow cell using SIR adapter design; the SIR being 20 nucleotides in length. SARS-COV-2 nucleic acid was detected, and PhiX was used as a positive control. This was the analysis used to generate the results for the SIR in FIGS. 6 and 7. The object specific python parser was used as an exemplary method for detecting each cluster by the signal from sequencing the SIR, and thereby distinguishing the signal from sequencing the target for each cluster from the signals from sequencing the target for all other clusters. In the alternative, the object specific python parser is used for identifying the presence of the first target in a sample by the presence of the first signal for the first SIR identifying said sample and the presence of the second signal that is at the same location as the location of the first signal for the first SIR identifying said sample.

FIG. 10 depicts sample SIRs (sample barcodes, SEQ ID NO: 9), forward and reverse target primers (SEQ ID NOS: 10 & 11, respectively) for detecting SARS-COV-2 nucleic acid, recognition sequences for native and synthetic (negative control) SARS-COV-2 (SEQ ID NOS: 12 & 13, respectively).

FIG. 11 depicts sample SIRs (sample barcodes, SEQ ID NO: 14), forward and reverse target primers (SEQ ID NOS: 15 & 16, respectively) for detecting Influenza A nucleic acid, recognition sequences for native and synthetic (negative control) Influenza A (SEQ ID NOS: 17 & 18, respectively).

FIG. 12 depicts sample SIRs (sample barcodes, SEQ ID NO: 19), forward and reverse target primers (SEQ ID NOS: 20 & 21, respectively) for detecting Influenza A nucleic acid, recognition sequences for native and synthetic (negative control) Influenza A (SEQ ID NOS: 22 & 23, respectively).

FIG. 13 depicts a Poisson regression count distribution that follows the centralized expected mean and variance, where the over dispersion is not observed in 96% of the barcode counts with average depth is around 1800±700 with 500 synthetic RNA copies and 1000 S2 gene copies.

FIG. 14A-C depicts tapestation results from Saliva Pre-Heat Assay showing maximum PCR amplification product at 15 minutes according to statistical analysis. Neat saliva diluted 1:1 with water, TE, PBS or 0.9% Saline is an effective way to increase reliability of saliva in the NGS Surveillance Assay.

FIG. 15 depicts the optimization of the validation of the unique set of barcodes that were performed using two Illumina platforms, MiSeq v2.6 and NextSeq 2000.

FIG. 16 depicts the equipment and manpower requirements for 10K samples processed in 24 hours.

FIG. 17 depicts heat inactivation of saliva

FIG. 18 depicts sample SIRs (sample barcodes, SEQ ID NO: 24), forward and reverse target primers (SEQ ID NOS: 25 & 26, respectively) for detecting SARS-COV-2 nucleic acid, recognition sequences for native and synthetic (negative control) SARS-COV-2 (SEQ ID NOS: 27 & 28, respectively).

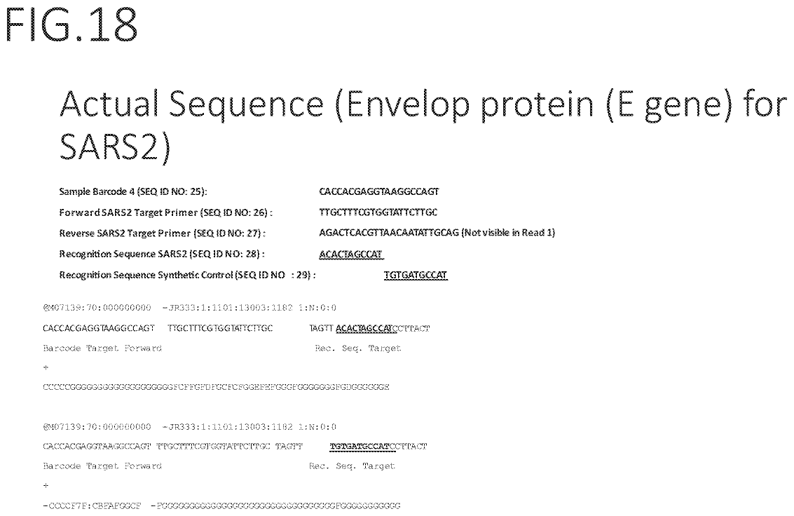

### DETAILED DESCRIPTION

### Definitions

The preferred materials and methods are described herein; any methods and materials similar or equivalent to those described herein can be used in the practice of or testing of the invention. Unless defined otherwise, all technical and scientific terms herein have the same meaning as commonly understood by one of ordinary skill in the art to which the invention pertains. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Unless otherwise indicated, “or” encompasses “and.” To illustrate, “A, B, or C” means A alone, B alone, C alone, the combination of A and B, the combination of A and C, the combination of B and C, and the combination of A, B, and C, unless otherwise illustrated.

“About” as used herein when referring to a measurable value such as an amount, a temporal duration, a quantum of measurement, and the like, is meant to encompass variations of .+−. 20% or .+−. 10%, more preferably .+−. 5%, even more preferably .+−. 1%, and still more preferably .+−. 0.1% from the specified value, as such variations are appropriate to perform the disclosed methods.

“Sequence” or “region” as used herein within the context of a nucleic acid, unless otherwise specified, includes sense and anti-sense (e.g., complementary) sequences of the same nucleic acid. To illustrate, if a specific sequence, called “A”, is 5′-ATGG-3′ in the sense strand then A also comprises 3′-TACC-5′ in the antisense strand (i.e., A comprises 5′-ATGG-3′ or 3′-TACC-5′). “Sequence” as used herein, unless otherwise specified, also includes different nucleic acids, i.e., RNA and DNA, of the same information (i.e., the information being the order of nucleotides in the sequence, e.g., genetic information), as well as sense and anti-sense (e.g., complementary) information therein. To illustrate, if A in RNA (sense) is 5′-AUGG-3′, A also comprises 5′-ATGG-3′, being the sense DNA, and 3′-TACC-5′ being the anti-sense DNA, as well as 3′-UACC-5′, being the antisense RNA. To distinguish between sense and anti-sense (e.g., complementary) sequences, a prime symbol (′) may be used, i.e., for case of tracking original genomic material, transcripts, first strand synthesis, second strand synthesis, sense, and anti-sense strands. To further illustrate, if a first single-stranded nucleic acid binding region comprises SEQ ID NO: 1, 5′-AATGATACGGCGACCACCGA-3′, then that first single-stranded nucleic acid binding region also comprises 5′-TCGGTGGTCGCCGTATCATT-3′, SEQ ID NO: 24 (i.e., first single-stranded nucleic acid binding region comprises SEQ ID NO: 1 or SEQ ID NO: 24). By this definition, any first adapter or second adapter enclosed may be provided in its complementary form to provide for adapted first strand and adapted second strand synthesis using the adapters directly on the mRNA or cDNA, and for using random primers for reverse transcription or first strand synthesis.

“Sample Identifying Sequence” also known as “Sample Identifying Region” (SIR) in some embodiments distinguishes or identifies one sample from all others in the assay by providing a unique nucleotide sequence in the adapter designated for that sample. In some embodiments, it is understood that although a sequence comprises the sense and anti-sense sequences (i.e., complementary), the sense and anti-sense sequences can be distinguished throughout the synthesis of subsequent strands (i.e., a first single-stranded product, a first single-stranded cDNA, a second single-stranded product, a second single-stranded cDNA). Thereby, a sample identifying region identifying one sample can be distinguished from a complementary sample identifying region identifying another sample.

In some embodiments, multiple targets are detected, and there is a first and second sample identifying region in the adapters, the adapters being specific for one target. In some embodiments, the first and second sample identifying regions are the same for that sample, and in some embodiments they are different. In some embodiments, a sample may be from one individual (a.k.a. subject), and in some embodiments, one individual may have multiple samples (i.e., obtained from sputum, blood, nasal swabs, or oral swabs). In some of the embodiments, the signals (e.g., fluorescent signal) obtained from sequencing the sample identifying regions provide for distinguishing one cluster from another cluster in the next generation sequencing flow cell and for pooling the signals from discrete clusters that share the same sample identifying regions.

Since clusters may overlap in the flow-cell, in some embodiments, the signals obtained from sequencing the sample identifying regions can be used to dis-intercalate overlapping signals from two or more clusters or to distinguish areas within a cluster that do not overlap another cluster (i.e., a cluster from another subject) (i.e., to distinguish areas where the signal is only from one cluster and therefore from one subject from areas in which two or more clusters overlap and therefore the signal is from two or more subjects). Since the signals obtained by next generation sequencing machines are electromagnetic waves (i.e., fluorescent signals) released from a location on the flow cell and since the resolution is limited by the optical characteristics of the imaging machinery of the next generation sequencing machines and by the theoretical minimum of half the wavelength of the emitted light, in some embodiments the signals obtained by sequencing the sample identifying regions, are used to identify an area of signal in on the flow cell that is discrete from all other areas of signal in the flow cell thereby distinguishing the arca of signal from one subject from the area of signal from another or all other subjects. In some of the embodiments, the signals obtained from sequencing the sample identifying regions provide for distinguishing signals obtained from sequencing a target from background signals, or for distinguishing specific DNA synthesis from non-specific or background DNA synthesis.

In some embodiments, the subject identifying sequence comprises at least 10, at least 11, at least 12, at least 13, at least 14, at least 15, at least 16, at least 17, at least 18, at least 19, at least 20, at least 21, at least 22, at least 23, at least 24, or at least 25 nucleotides. In some embodiments the subject identifying sequence comprises 10, 11, 12, 13, 14, 15, 16, 17, 18, 19, 20, 21, 22, 23, 24, 25, 26, 27, 28, 29, or 30 nucleotides. In some embodiments the subject identifying sequence comprises no more than 50, no more than 45, no more than 40, no more than 35, no more than 30, or no more than 25 nucleotides. In some embodiments, the subject identifying sequence comprises one or more redundancies in its nucleotide sequence to provide for correction of one or more, two or more, three or more, four or more, five or more mismatches in the sequencing of the subject identifying sequence without disqualifying the sample from identification.

As noted above, the SIR identifies a sample by providing a unique nucleotide sequence for that one sample. In some embodiments, the SIR comprises at least 80,000 unique nucleotide sequences that can be detected with the sequencing machine. In some embodiments, the SIR comprises at least 67,000 unique nucleotide sequences that can be detected with the sequencing machine.

In some embodiments, the method comprises 100 or more, 200 or more, 300 or more, 400 or more, 500 or more, 600 or more, 700 or more, 800 or more, 900 or more, 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, or 8500 or more samples. In some embodiments, the method comprises less than 100, less than 200, less than 300, less than 400, less than 500, less than 600, less than 700, less than 800, less than 900, less than 1000, less than 1500, less than 2000, less than 2500, less than 3000, less than 3500, less than 4000, less than 4500, less than 5000, less than 5500, less than 6000, less than 6500, less than 7000, less than 7500, less than 8000, less than 8500, less than 9000, less than 10000, less than 11000, less than 12000, less than 13000, less than 15000, less than 20000, less than 25000, less than 30000, less than 40000, less than 50000, less than 60000, less than 67000, less than 70000, less than 75000, or less than 85000 samples.

“Substrate” as used herein, and unless otherwise specified, refers to the solid-state support of a flow cell. The term may in generic chemical reactions refer to the reactant, or to the reactant in an enzyme system.

“Nucleic acid,” “polynucleotide,” and “oligonucleotide” as used herein all have the same meaning and they are composed of a sequence of nucleotides, each nucleotide comprising a phosphate and a nucleoside, a nucleoside comprising a pentose sugar (e.g., deoxyribose and ribose) and a nucleobase (e.g., a purine comprising adenine or guanine and a pyrimidine comprising cytosine, uracil, and thymine).

The term “pathogen” as used herein refers to a bacteria, virus, fungus or parasite that is capable of infecting and/or causing adverse symptoms in a subject.

The term “sample” or “biological sample” means biological material isolated from a subject. The biological sample may contain any biological material suitable for detecting the desired target, and may comprise cellular and/or non-cellular material from the subject. Typical examples of biological material include but are not limited to urine, blood, plasma, tissue homogenate, tears, saliva, vaginal fluid, semen, fecal sample, upper respiratory mucus, breath condensate, wound discharge and spinal fluid.

### Kit

According to certain embodiments, provided is a kit for detecting in vitro the presence or absence of one or more targets in a sample. The targets typically pertain to a single strand of nucleic acid, typically, but not necessarily, from a pathogen.

In a specific embodiment, the kit includes the following components: 1) a master mix, 2) for each target, a first adapter and a second adapter, and 3) a positive control specific for each target. In various embodiments, the pathogen is a virus. Examples of viruses include but not limited influenza, coronavirus, arenavirus, a filovirus, alphavirus, hantavirus, and a flu (e.g., influenza) virus. In some embodiments, the hantavirus includes Andes virus, Sin Nombre virus, Hantaan virus, and Puumala virus. In some embodiments, the arenavirus includes Junin virus, Machupo virus, Guanarito virus, and Sabia virus. In some embodiments, the filovirus includes cuevavirus, dianlovirus, ebola virus and Marburg virus. In some embodiments, the ebola virus includes Bombali virus, Bundibugyo virus, Reston virus, Sudan Virus, Tai Forest Virus, and Zaire ebolavirus. In some embodiments, the pathogen is from members of the kingdom Orthornavirac, or members from the phylum Negarnaviricota, or members from the class Insthoviricetes, or members from the order Articulavirales, or members of the family Orthomyxoviridae, or members from the genera Alphainfluenzavirus including Influenza A, Betainfluenzavirus including Influenza B, Deltainfluenzavirus including Influenza D, or Gammainfluenzavirus including influenza C. In some embodiments, the Influenza A is HIN1, H2N2, H3N2, H5N1, H7N7, HIN2, H9N2, H7N2, H7N3, or H10N7. In some embodiments, the coronavirus is SARS-COV, SARS-COV-2, human coronavirus OC43, human coronavirus HKU1, human coronavirus 229E, human coronavirus NL63, or Middle East respiratory syndrome-related coronavirus (MERS-COV).

In other embodiments, the pathogen is a a bacterium. Examples of bacteria include but not limited to Bacillus, Bacteroides, Bartonella, Bordetella, Brucella, Burkholderia, Campylobacter, Chlamydia, Clostridium, Corynebacterium, Enterococcus, Escherichia coli, Haemophilus, Lactobacillus, Mycobacterium, Mycoplasma, Neisseria, Pasteurella, Rickettsia, Salmonella, Staphylococcus, Streptococcus, Treponema, Vibrio, Wolbachia, or Yersinia. In some embodiments, the pathogen may consist of a fungus. Examples of fungi include but not limited to Blastomyces, Cryptococcus, Coccidioides, Histoplasma, Aspergillus, Pneumocystis, Candida, Mucorales, or Talaromyces.

In certain embodiments, the master mix comprises a reaction buffer, a polymerase, and dNTPs. In a specific embodiment, the master mix comprises Platinum™ Taq DNA Polymerase (Thermo Fisher Scientific) with the following recipe, 2× Reaction Buffer, polymerase, and DEPC water. In certain embodiments of the kit, the first adapter includes from 5′ to 3′ a first primer binding region, a first sample identifying region (SIR), and a first target primer, each first SIR comprises a sequence that identifies each sample, the second adapter comprises from 5′ to 3′ a second primer binding region and a second target primer. In certain embodiments, the first primer binding region comprises SEQ ID NO. 5 and the second primer binding region comprises SEQ ID NO. 6

In a specific embodiment, the target is SARS-COV-2 with the first target primer comprising SEQ ID NO. 7 and the second target primer comprising SEQ ID NO. 8.

When a kit is ordered the physician or consumer may specify the target such that the adapters of the kit include primers to the specified target. Also, the SIR will be specific to the kit, which will be used to associate the subject from which the sample tested is obtained.

### Methods

Provided herein are methods of detecting in vitro the presence or absence of a target in a sample; the method comprising detecting the target in two or more samples. In some embodiments, the first target is a single strand of nucleic acid. In some embodiments, the target is from a pathogen, such as a virus or a bacterium, infecting a subject. In some embodiments, the subject is diagnosed as being infected by the presence or absence of the target in a sample taken from the subject. In some embodiments, the presence or absence of target is detected in two or more samples, and thereby more than two subjects are diagnosed as being infected or not infected. Since double-stranded DNA and double-stranded RNA are composed of single-stranded DNA and single-stranded RNA, in some embodiments, the target is isolated to obtain a single-stranded nucleic acid. In some embodiments, the target is isolated from the sample prior to detection. For example, if the target is a single-stranded nucleic acid, and if the pathogen has a genome comprising a double-stranded nucleic acid (i.e., a double-stranded RNA as in some viruses, or a double-stranded DNA as in the genome of bacteria), then the double-stranded nucleic acid can be denatured, or fragmented and denatured, to obtain a single-stranded nucleic acid. In some embodiments, the single-stranded nucleic acid can be single-stranded RNA. In some embodiments, the method comprises reverse transcription to make the target (i.e., the information contained in the single-stranded RNA) more indelible, because RNA is more at risk of degradation than DNA.

In some embodiments, the target is from a virus or a bacteria. In some embodiments, the target is from double-stranded RNA, double-stranded DNA, genomic nucleic acids, from mRNA, or from micro-RNA. In some embodiments, the target is from a virus that causes cold or flu-like symptoms, including but not limited to influenza, coronavirus, arenavirus, a filovirus, alphavirus, hantavirus, and a flu (e.g., influenza) virus. In some embodiments, the hantavirus includes Andes virus, Sin Nombre virus, Hantaan virus, and Puumala virus. In some embodiments, the arenavirus includes Junin virus, Machupo virus, Guanarito virus, and Sabia virus. In some embodiments, the filovirus includes cuevavirus, dianlovirus, ebola virus and Marburg virus. In some embodiments, the ebola virus includes Bombali virus, Bundibugyo virus, Reston virus, Sudan Virus, Tai Forest Virus, and Zaire ebolavirus. In some embodiments, the target is from members of the kingdom Orthornavirac, or members from the phylum Negarnaviricota, or members from the class Insthoviricetes, or members from the order Articulavirales, or members of the family Orthomyxoviridae, or members from the genera Alphainfluenzavirus including Influenza A, Betainfluenzavirus including Influenza B, Deltainfluenzavirus including Influenza D, or Gammainfluenzavirus including influenza C. In some embodiments, the Influenza A is HIN1, H2N2, H3N2, H5N1, H7N7, HIN2, H9N2, H7N2, H7N3, or HION7. In some embodiments, the coronavirus is SARS-COV, SARS-COV-2, human coronavirus OC43, human coronavirus HKU1, human coronavirus 229E, human coronavirus NL63, or Middle East respiratory syndrome-related coronavirus (MERS-COV).

In some embodiments, the target is any known reference gene, transcript, exon, intron, micro-RNA, or isolate thereof, which is to be detected in the population or subset thereof. In some embodiments, the method comprises designing two or more target primers, which may be included in the adapters or which may be used as primers for a pre-amplification or pre-isolation of the target. The identification of two or more target primers is the same analysis as is used for designing two or more primers for the isolation or amplification of the target of interest, provided that the product being isolated or amplified is of a length that can be detected in a next generation sequencing machine that sequences by synthesis. Because of the likelihood in jumps or delays in nucleotide addition during the sequencing-by-synthesis processes, the error rate generally increases as the target length increases. It is thereby preferred that the target-binding-regions bind to the target to isolate or amplify, with the target primers included, no more than 1000 nucleotides, preferably, no more than 500 nucleotides, preferably still no more than 300 nucleotides. Generally, the preferred length of the target being isolated, with the length of the target primers included, be that that in semi-qPCR, qPCR, conventional PCR, or reverse transcription methods, or is preferably at least 30, 40, 50, 60, 70, 80, 90, or 100 nucleotides in length. The target primers may be designed using any primer design program for conventional PCR, semi-qPCR, qPCR, or reverse transcription provided that the reference gene, transcript, intron, or micro-RNA provides for an amplicon having the above-noted nucleotide lengths.

In some embodiments, the method comprises adding at least two primer binding regions and at least one sample identifying region (SIR) to the target. In some embodiments, on one end (i.e., 5′ end or 3′ end) of the target will be one of the at least two primer binding regions and the SIR and on the other end will be the other of the at least two primer binding regions. To illustrate, in some embodiments, the method comprises a first reaction mixture comprising a first adapter and a second adapter. In some embodiments, the first adapter comprises from 5′ to 3′ a first primer binding region, a first sample identifying region (SIR), and a first target primer. In some embodiments, each first SIR comprises a sequence that identifies each sample. In some embodiments, the second adapter comprises from 5′ to 3′ a second primer binding region and a second target primer. In some embodiments, to the first primer binding region, a first primer (i.e., a first read primer for the sequencing-by-synthesis) or a first single-stranded nucleic acid anneals, depending upon the step in the method. In some embodiments, the first single-stranded nucleic acid is one of two single-stranded nucleic acid bound to the substrate of the flow cell used during at least one of the sequencing-by-synthesis and bridge amplification. In some embodiments, to the second primer binding region, a second primer (i.e., second read primer) or a second single-stranded nucleic acid anncals. In some embodiments, the second single-stranded nucleic acid is the other of the two single-stranded nucleic acids bound to the substrate (i.e., solid state-support) of the flow cell used during at least one of the sequencing-by-synthesis and bridge amplification. In some embodiments, the first and second single-stranded nucleic acids function as a universal first and second end, as in U.S. Pat. No. 7,985,565, or as a first- or second-flow cell recognition sequence. In some embodiments, the regions in the first primer binding region that bind the first read primer or the first single-strand nucleic acid, are the same, they overlap, or they are discrete. In some embodiments, the regions in the second primer binding region that bind the second read primer or the second single-strand nucleic acid, are the same, they overlap, or they are discrete.

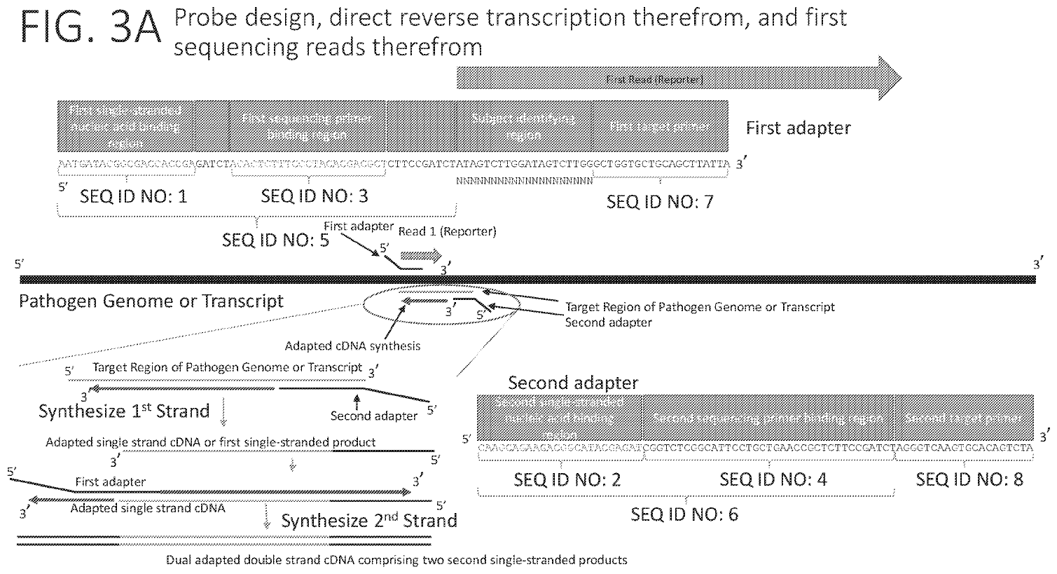

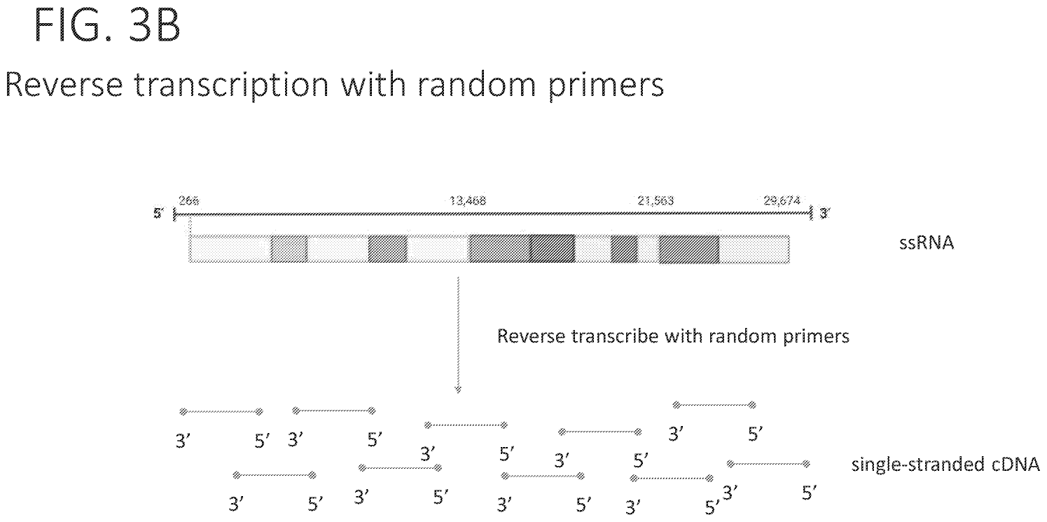

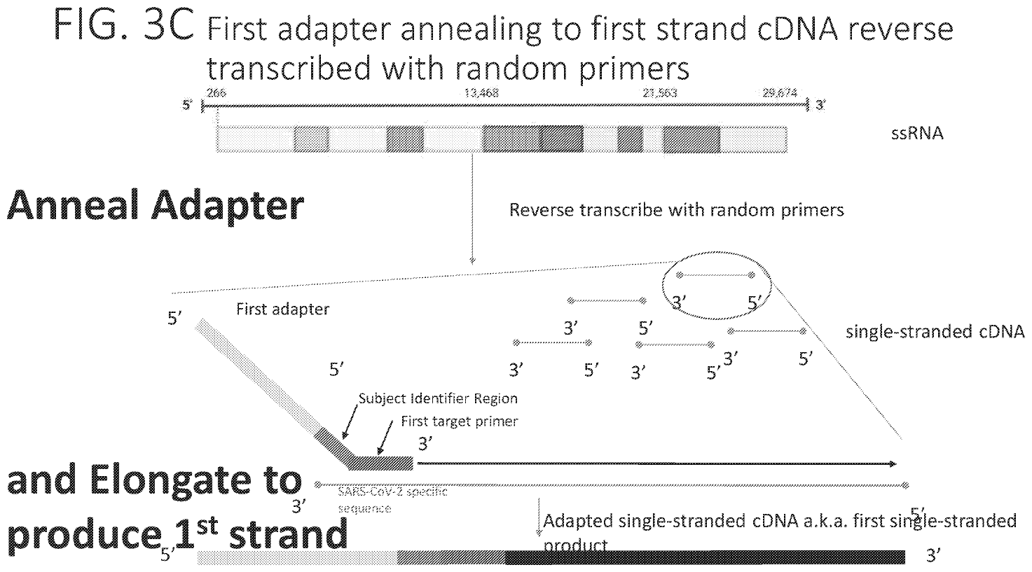

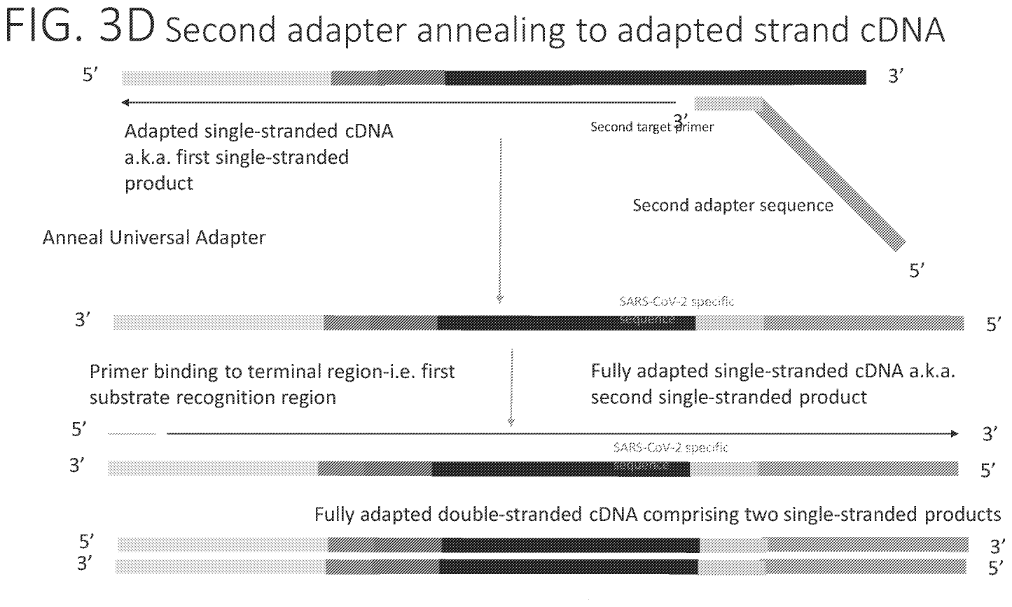

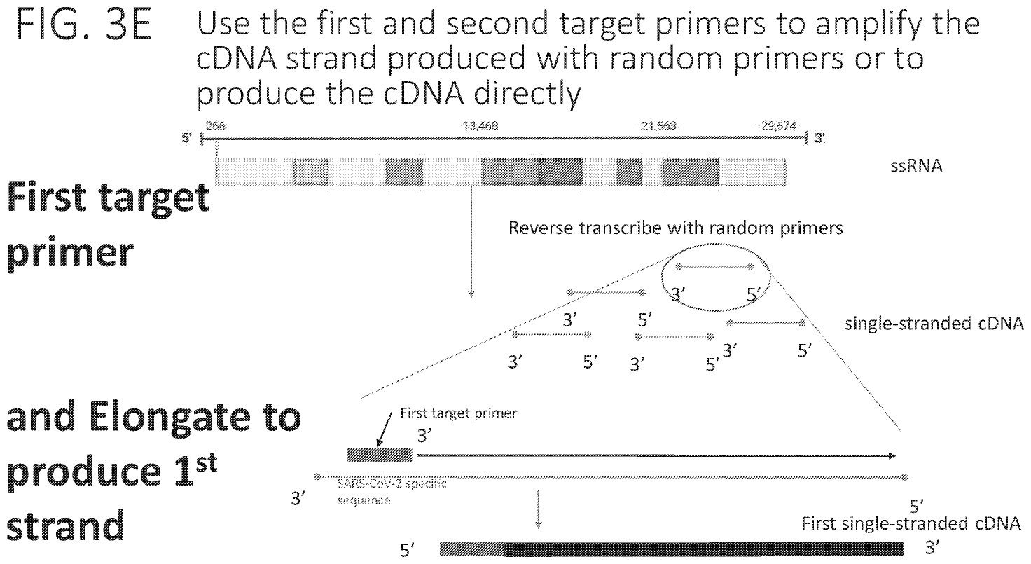

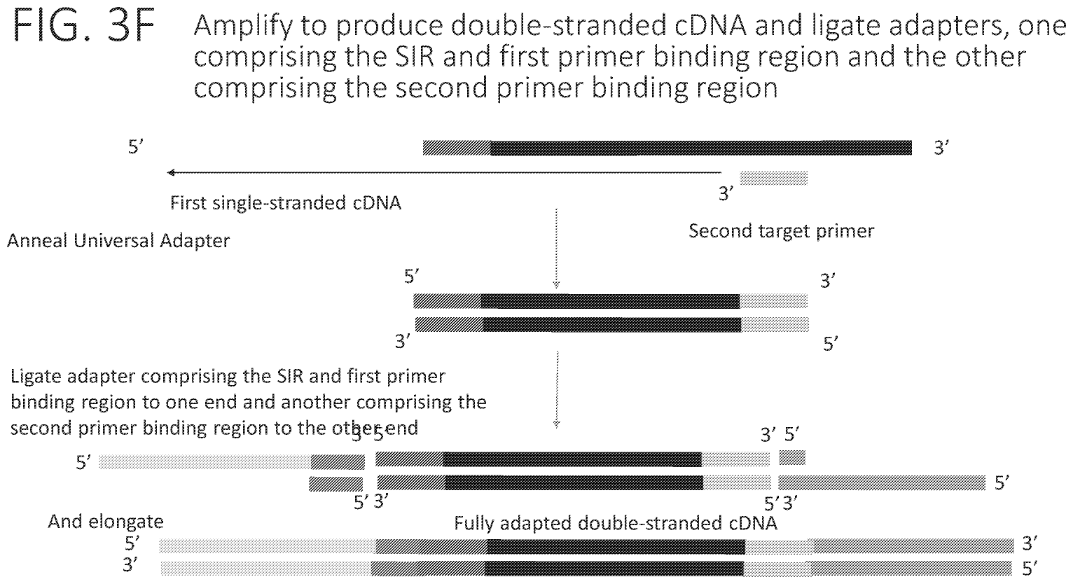

FIG. 3A illustrates one preferred embodiment where the first primer binding region comprises a first single-stranded nucleic acid primer binding region, which may anneal to the first single-stranded nucleic acid, and a first read primer binding region, which may anneal to the first read primer. FIG. 3A also illustrates a preferred embodiment where the second primer binding region comprises a second single-stranded nucleic acid primer binding region, which may anneal to the second single-stranded nucleic acid, and a second read primer binding region, which may anneal to the second read primer. In this embodiment, though illustrative, the first primer binding region and SIR is combined within an adapter (i.e., a first adapter) further comprising a first target primer, and the second primer binding region is comprised within an adapter (i.e., a second adapter) comprising the second target primer.

In some embodiments, the at least two primer binding regions and at least one SIR are added to the target by annealing or by ligating. To illustrate one method involving annealing, one adapter (i.e., a first adapter) comprising from 5′ to 3′ one primer binding region, the SIR, and one target primer will anneal to a region on the target, and the first adapter will be elongated, thereby adding to the 3′ end of the adapter the sequence of the target, thereby obtaining a first product.

In this embodiment, the target primer and the target anneal, thereby adding the one primer binding region and the SIR. After elongating, another adapter (i.e., second adapter) is annealed to the first product, wherein the second adapter comprises from 5′ to 3′ the other primer binding region and the second target primer. In this illustrative embodiment, the second primer binding region binds to a region of the target in the first product, and the second adapter is elongated, thereby obtaining a second product comprising the first primer binding region, the SIR, the target, and the second primer binding region. In this embodiment, the second target primer anneals to the target thereby adding the second primer binding region. In other embodiments, the second adapter comprises the SIR (i.e., the second adapter comprises from 5′ to 3′ the second primer binding region, the SIR, and the second target primer.

The naming of the first and second adapters, first and second primer binding region, and first and second target primers is not intended to convey the order of the binding or annealing. See FIGS. 3A-3F illustrating different ordering of the first and second adapters in the methods of annealing and elongating, depending upon how the target is initially processed. Therefore, “first,” “second,” “third,” and any other number is used to provide some nomenclature with which to refer to discrete oligonucleotide sequences, and the relationship of that oligonucleotide to other oligonucleotides to which they might anneal at particular steps in the method or to which they may be 3′ of or 5′ of in a particular oligonucleotide. I.e., first target and second target.

In some embodiments, the elongating in the first or second strand synthesis further comprises reverse transcription, such as when the target is initially a single or double-stranded RNA from a virus or mRNA from a bacterium or eukaryotic cell. Sec FIGS. 3B, 3C, and 3D. It is understood that double-stranded RNA comprises two single-stranded RNAs, and a double-stranded DNA comprises two single-stranded DNA. It is understood that in an annealing step, double-stranded nucleic acids are often denatured to produce two single-stranded nucleic acids, to at least one of which one or more primers (i.e., target primers within adapters) can bind. Accordingly, it is understood that in some embodiments, the methods disclosed herein can comprise, coextensive with any annealing or prior to any annealing, the isolation of a single-stranded nucleic acid target from a double-stranded nucleic acid target (i.e., by denaturing).

In some embodiments, one adapter and another adapter are annealed to the target. To each sample is added a first reaction mixture comprising a first adapter and a second adapter. The one adapter comprises from 5′ to 3′ a first primer binding region, a sample identifying region (SIR), and a first target primer. Each SIR comprises a sequence that identifies each sample. The second adapter comprises from 5′ to 3′ a second primer binding region and a second target primer. The admixing will be under conditions when the target is present in the sample wherein one of the adapters anneals to the target, wherein a one of the first target primer or the second target primer anneals to the first target, thereby obtaining a first target-bound adapter. The first target-bound adapter is elongated to obtain a first single-stranded product comprising: I) the target and Ila) the first primer binding region and first SIR or IIb) the second primer binding region. The other of adapters anneals to the first single-stranded product, wherein the other of the first target primer or the second target primer anneals to the first target, thereby obtaining a second target-bound adapter. And, the second target-bound adapter is elongated, thereby obtaining a second single-stranded product comprising the first primer binding region, first SIR, the first target, and the second primer binding region.

In some embodiments, adapters are added to the target. In some embodiment, the product of adding adapters to the target is to create a target comprising from one end to the other, one primer binding region (i.e., a first primer binding region or a third primer binding region), a SIR, the target, and another primer binding region (i.e., a second primer binding region or a fourth primer binding region, respectively). In some embodiments, target primers, functioning as primers, provide a means with which the other components i.e., the primer binding regions and the SIRs may be added to the target. In some embodiments, the adapters comprise a primer binding region and the SIR (i.e., a first primer binding region and a first SIR), or a primer binding region (i.e., a second primer binding region). In some embodiments, the target primers can be used alone as primers, and then the adapters comprising the primer binding region and the target primers, and further optionally, the SIR, may be used to further obtain the product comprising the first primer binding region, the SIR, the target, and the second primer binding region. Sec FIGS. 3B-3F. In the alternative, random primers may be used to amplify and/or reverse transcribe the target, then the adapters comprising the primer binding region and the target primers, and further optionally, the SIR, may be used to further obtain the product comprising the first primer binding region, the SIR, the target, and the second primer binding region. In both these embodiments, the initial use of random primers or target primers as primers can amplify the target (i.e., the region of the nucleic acid wishing to be detected, e.g., the a 50 nucleotide region of the SARS-COV-2 genome) prior to having the adapters ligated or annealed, and thereby prior to having the primer binding regions and the SIR added.

In some embodiments, the reaction mixture comprises a ligase, a DNA polymerase, an RNA polymerase (i.e., a transcriptase), or a reverse transcriptase.

In some embodiments, the amplification of the target using primers (i.e., target primers or random primers) can create a double-stranded target to which the adapters can be ligated. To illustrate, to each sample, a reaction mixture can be admixed. In some embodiments, the reaction mixture comprises the first target primer and the second target primer. In some embodiments, the admixing is under conditions when the target is present in the sample the target is amplified, thereby obtaining a double-stranded target. Further admixing is performed, admixing two adapters (i.e., a first and second polynucleotide) to the reaction mixture. One adapter (i.e., a first polynucleotide) comprises the first primer binding region and the SIR. The other adapter (i.e., the second polynucleotide) comprises the second primer binding region. The one adapter and the other adapter are ligated to the double-stranded target. In some embodiments, the ligation can occur by creating a single nucleotide overhang (i.e., an A) on each end of the double-stranded nucleic acid, and the adapters can comprise a single nucleotide overhang (i.e., a T) allowing for the proper orientation.

In some embodiments, one adapter (e.g., the adapter comprising the first primer binding region and the SIR) is ligated to an opposite end of the double-stranded target as the other adapter (e.g., the adapter comprising the second primer binding region). In the ligating, the SIR is proximal and the first primer binding region is distal to the target; thereby at least a double-stranded product is obtained, wherein the product comprises, in order, the first primer binding region, the SIR, the target, and the second primer binding region. Since this product is double-stranded, it can be denatured, to obtain a single-stranded product comprising, in order, the above-noted regions. In one strand, the orientation of these above-stated components is 3′ to 5′. In the other strand, the orientation of the above-stated components is 5′ to 3′. Since the first and second primer binding regions are annealed respectively to the first and second nucleic acids of the flow cell, in some embodiments, only one of the two single-stranded products is used. Only one of the two single-stranded products will have the correct orientation to be bridge amplified on the flow cell.

In alternative embodiments, the adapters are set up as Y adapters. A Y-adapter comprises a double-stranded region and two arms, one arm not being hybridizable to the other arm. One arm would comprise comprising the first primer binding region and first SIR, and the other arm would comprise the second primer binding region. The Y-adapter would be ligated to both ends of the double-stranded nucleic acid, and then it would be amplified by using the first and second primer binding regions, or portions thereof (e.g., unbound first and second single-stranded nucleic acids), as primers.

In some embodiments, the admixture of the reaction mixture to each sample provides a modified sample for each sample. In some embodiments, the first modified samples are pooled, thereby obtaining a pooled sample. In some embodiments, the pooled sample is then flowed over the flow cell.

In some embodiments, the flow cell comprises a substrate, a first single-stranded nucleic acid, and a second single-stranded nucleic acid. In some embodiments, the 5′ end of each of the first single-stranded nucleic acid and the second single-stranded nucleic acid is bound to the substrate. In some embodiments, the first single-stranded nucleic acid is capable of a first annealing to the first primer binding region. In some embodiments, the second single-stranded nucleic acid is capable of a second annealing to the second primer binding region. In some embodiments, the flowing is under conditions that permit at least one of the first annealing or the second annealing; thereby obtaining a single-stranded product that is annealed to the flow cell.

In some embodiments, the method further comprises bridge-amplifying the single-stranded product that is annealed to the flow cell; thereby obtaining two or more clusters. In U.S. Pat. No. 7,985,565, bridge-amplifying creates colonies, and “clusters” herein are understood to have the same meaning as “colonies.” In some embodiments, each cluster comprises a second primer binding region-bound single-stranded product and has a location on the flow cell. In some embodiments, the second primer binding region-bound single-stranded product comprises the single-stranded product wherein the 5′ end of the second primer binding region is bound to the substrate. In some embodiments, each second primer binding region-bound single-stranded product in each cluster has the same SIR and thereby is from the same sample.

It is understood that the first and second single-stranded nucleic acids of the flow cell and the first and second primer binding regions are named first and second not to indicate the order in which they bind but to assign a label to indicate to which the other binds. For example, the first primer binding region binds to the first single-stranded nucleic acid. And for example, the second primer binding region binds to the second single-stranded nucleic acid. The first primer binding region may be annealed or ligated to the target after the second primer binding region, depending upon the order of and methods for the above-noted annealing or ligating processes.

The process of sequencing-by-synthesis of at least a portion of the target and the SIR will be described. Generally, though not required, the bridge-amplifying creates two products, one in which the first primer binding region is bound at its 5′ end to the substrate, and another in which the second primer binding region is bound to its 5′ end of the substrate. To avoid non-specific sequencing-by-synthesis, generally one of these two strands is cleaved by using a specific nuclease that targets one of the first single-stranded nucleic acids or the second single-stranded nucleic acids. In some embodiments, the nuclease targets either the first or the second single-stranded nucleic acid at the 3′ terminal of the first or second single-stranded nucleic acid. That is, when the first or second single-stranded nucleic acid are elongated, the new strand will comprise a 5′ bound first or second primer binding region, and the nuclease will cleave the first or second primer binding region at or near the 3′ end of the first or single-stranded nucleic acid where the elongation is initiated. In this regard, the first or second primer binding region may comprise within it a region where the first or second single-stranded nucleic acid anneals, and the nuclease will cleave the first or second primer binding region at the boundary between the region where the first or second single-stranded nucleic acid anneals and the rest of the first or second primer binding region.

Since new strands of nucleic acids are polymerized in a 5′ to 3′ direction based off of reading a template in a 3′ to 5′ direction, in a preferred embodiment, the strands produced by bridge-amplifying in which 5′ end of the first primer binding region is bound to the substrate are cleaved by the nuclease, thereby retaining the strands in which the 5′ end of the second primer binding region is bound to the substrate. Accordingly, this strand will have the first primer binding region at the 3′ end, and the 3′ end will not be linked to the substrate. In some embodiments, at least one of a first read primer (a.k.a. a first sequencing primer) can anneal to the first primer binding region. In some embodiments, the elongation of the first sequencing primer initiates sequencing-by-synthesis of the first read. In some embodiments, the first read sequencing primer can comprise two or more primers (i.e., a first sequencing primer binding to the first primer binding region that initiates the sequencing by synthesis of the SIR and a third sequencing primer that initiates the sequencing of the target). Thereby, in some embodiments, the first adapter comprises a third sequencing primer binding region downstream of the SIR but upstream of the target. In some embodiments, at least one of a second read primer (a.k.a. a second sequencing primer) can anneal to the second primer binding region. In some embodiments the elongation of the second sequencing primer initiates sequencing-by-synthesis of the second read. In some embodiments, the second read sequencing primer can comprise two or more primers (i.e., a second sequencing primer that initiates the sequencing by synthesis of a third SIR, when the second adapter comprises a SIR (e.g., a third SIR), and a fourth sequencing primer that initiates the sequencing of the target). Thereby, in some embodiments, the second adapter comprises a third sequencing primer binding region downstream of the SIR but upstream of the target. In some embodiments, the SIR will be read, or sequenced-by-synthesis before the target. That is, the first sequencing primer will anneal to the template in the 3′ direction of the SIR. In some embodiments, the first sequencing primer will be or have the same sequence as the first single-stranded nucleic acid. In some embodiments, the first sequencing primer will be the first single-stranded nucleic acid that is bound at its 5′ end to the substrate. In some embodiments, the first sequencing primer comprises the first primer binding region (i.e., be the complementarity of the entirety of the first primer binding region). In some embodiments, the primer binding region will comprise a single-stranded nucleic acid binding region and a sequencing primer binding region. In some embodiments, the sequencing primer will bind to the sequencing primer binding region. In some embodiments, the single-stranded nucleic acid will bind to the single-stranded nucleic acid binding region.

In some embodiments, the sequencing-by-synthesis comprises a four fluorophore signal (one for each nucleotide). In another embodiment, the sequencing-by-synthesis comprises a two fluorophore/color signal, wherein one of the nucleotides (i.e., T) is labeled one color, another nucleotide (i.e., C) is labeled another color, a third nucleotide (i.e., A) is labelled with both colors, and wherein one base (i.e., G) is not associated with a fluorescent/color signal. In some embodiments, the sequencing-by-synthesis comprises a one fluorophore/color signal, wherein two nucleotides are labeled with a fluorophore or color (i.e., A and T) and wherein one of the two nucleotides can have the fluorophore or color cleaved (i.e., A), and wherein another of the nucleotides has a group to which another fluorophore or color of the same color can bind (i.e., C).

In some embodiments, the first read of the sequencing-by-synthesis sequences the SIR before the target or before at least a portion of the target. In some embodiments, the sequencing-by-synthesis of the target is in-line and/or downstream of the sequencing-by-synthesis of the SIR. In some embodiments, the first sequencing primer initiates the sequencing-by-synthesis of the SIR alone. In some embodiments, the first sequencing primer initiates the sequencing-by-synthesis of the target alone. In some embodiments, the first sequencing primer initiates the sequencing-by-synthesis of the SIR and the target, the target being sequenced in-line and/or downstream of the SIR.

In some embodiments, the sequencing-by-synthesis sequences the entirety of the target in the first read (i.e., in situations where the target is relatively short, i.e., around 50 nucleotides). In some embodiments, the sequencing-by-synthesis of the first read sequences at least a first region within the target, the first region being proximal to the SIR. In some embodiments, the sequencing-by-synthesis of the second read sequences at least a second region within the target, the second region being proximal to the second primer binding region. In some embodiments, the first region and second region within the target comprises the entirety of the target; thereby the signals obtained from sequencing the first region within the target and the signals obtained from sequencing the second region within the target comprise the entirety of the signals of the target and thereby the entire sequence of the target. In some embodiments, a sequence that overlaps between the first and second regions within the target are used to compile the first and second target reads, thereby obtaining the sequence of the target.

In some embodiments, the sequencing-by-synthesis of the first SIR and at least a first sequence within the target generates a plurality of first read signals. In some embodiments, the plurality of first read signals comprises a plurality of a one signal (i.e., first signals) and a plurality of another signals (i.e., second signals). In some embodiments, each of the one and another signals (i.e., each of the first signals and each of the second signals) has a location on the flow cell. In some embodiments, the one signal (i.e., first signal) comprises the signal from sequencing the SIR, and the other signal (i.e., second signal) comprises the signal from sequencing the at least a first sequence within the target, or the entirety of the target. In some embodiments, the first read signals further comprise a first background signal. In some embodiments, the first background signal is from locations on the flow cell not having a cluster.

In some embodiments, the sequencing-by-synthesis comprises sequencing two or more targets. In some embodiments the two or more targets are obtained using the above-noted steps, wherein one target is modified to have the first target primer, a first SIR, the one target, and the second primer binding region, and another target is modified to have the first primer binding region, a second SIR, the one target, and the second primer binding region. In some embodiments, the first and second SIRs can be the same, or in other embodiments, the first and second SIRs might be different. In the bridge-amplifying, each cluster comprises the second primer binding region-bound one single-stranded product or a second primer binding region-bound other single-stranded product. Each of the second primer binding region-bound one single-stranded product and the second primer binding region-bound other single-stranded product will be bound at 5′ end of the second primer binding region to the substrate. Each of the second primer binding region-bound one or other single-stranded product in each cluster will have the same SIR and thereby be from the same sample.

In the sequencing-by-synthesis of two or more targets, the second SIR and at least one sequence within the other target is sequenced from the elongated end of the first sequencing primer. In some embodiments, the at least one sequence within the second target is proximal to the second SIR. In some embodiments, there will be a second read wherein at least another sequence within the other target is sequenced-by-synthesis. After the first read, the elongated product of the first read may be washed away. In some embodiments, the second primer binding region-bound single-stranded product is retained. The first primer binding region of the second primer binding region-bound single-stranded product is annealed to the first single-stranded nucleic acid, and the first single-stranded nucleic acid is elongated, thereby obtaining a first primer binding region-bound single-stranded product comprising the single-stranded product wherein the 5′ end of the first adapter is bound to the substrate. In some embodiments, a nuclease then cleaves second primer binding region-bound single-stranded product. Thereby, each cluster thereby comprises the first primer binding-region bound single-stranded product. Each cluster thereby has single-stranded products having the same SIR, and thereby being from the same sample.

In some embodiments, a second sequencing primer is annealed to the second primer binding region of the first primer binding region-bound second single-stranded product. In some embodiments, the second sequencing primer is elongated, sequencing-by-synthesis at least a second sequence within the first target. In some embodiments, the second sequence within the first target is proximal to the second primer binding region. Thereby, a plurality of second read signals is obtained comprising a plurality of other signals (i.e., third signals). Each of the other signals (i.e., each of the third signals) has a location on the flow cell, and the other (i.e., third) signal comprises the signals from sequencing the at least the second sequence within the first target. In some embodiments, the one sequence (i.e., first sequence) within the target together with the other sequence (i.e., second sequence) within the first target comprises the first target (i.e., the entirety of the first target).

The following is a description of the analysis of the signals obtained from the first and/or second read (i.e., the first read signals and the second read signals). In some embodiments, a library is generated. In some embodiments, a cluster-differentiated library is generated. In some embodiments, a cluster-differentiated-by-SIR library is generated.

In some embodiments, the one sequence and other sequence within the other target will together comprise the other target. In the sequencing-by-synthesis of two or more targets, the plurality of first read signals further comprises a plurality of fourth signals and a plurality of fifth signals. Each of the fourth signals and each of the fifth signals will have a location on the flow cell. In some embodiments, the fourth signal comprises the signal from sequencing the second SIR. In some embodiments, the fifth signal comprises the signal from sequencing the at least the third sequence within the second target. This construction can continue for additional targets, i.e., a sixth signal for detecting a third SIR and an seventh signal for detecting at least a portion of the next target. The numeration of the signals is only intended to distinguish the reads from one adapted target from another adapted target. Each adapted target comprises a first and second primer binding region and a SIR.

In some embodiments, the method further comprises identifying the presence or absence of the one or more targets in the sample. In some embodiments, the presence of one target in the sample is identified by the presence of the signal for the SIR identifying said sample and the presence of the signal for that target that is at the same location as the location of the signal for the SIR identifying the sample, and the absence of the that target in the sample is identified by the absence of the signal for the SIR identifying said sample or the absence of the signal for that target at the same location as the location of signal for the SIR identifying said sample.

In generating a cluster-differentiated-by-SIR library, in some embodiments, the generating comprises identifying the location of each cluster on the flow cell by the location of the signal obtained by sequencing the SIR (i.e., identifying each cluster as being distinct from the next because each cluster came from a different sample). Where the method comprises two or more targets (i.e., a first and a second SIR), in some embodiments, the location of each cluster will be identified by the location of the SIRs (i.e., the location of the first SIR or the location of the second SIR). By identifying the location of each cluster by the location of each SIR, it is thereby easier to distinguish the signal from sequencing a target from one cluster from the signal from sequencing a target from all the other clusters. That is, one advantage of this method is that where all the targets are the same (i.e., all are SARS-COV-2) and where traditional next generation sequencing distinguishes signals from one another by differences in the target sequence (i.e., one fragment of genomic DNA has a different sequence than another fragment of genomic DNA), the next generation sequencing machine, algorithms, and software might treat several clusters each from a different sample as being the same cluster or same information because each of these clusters have the same target sequence. By distinguishing the clusters, and thereby the signals from each sample, by the location of the signal sequencing the SIR, the method is able to thereby distinguish each signal from sequencing the target from the next by the location of the signal from sequencing the SIR. Accordingly, in some embodiments, the generating comprises distinguishing the signal from sequencing the target at one location on the flow cell within the plurality of signals from sequencing the target at all the locations on the flow cell by the location of the signal from sequencing the SIR.

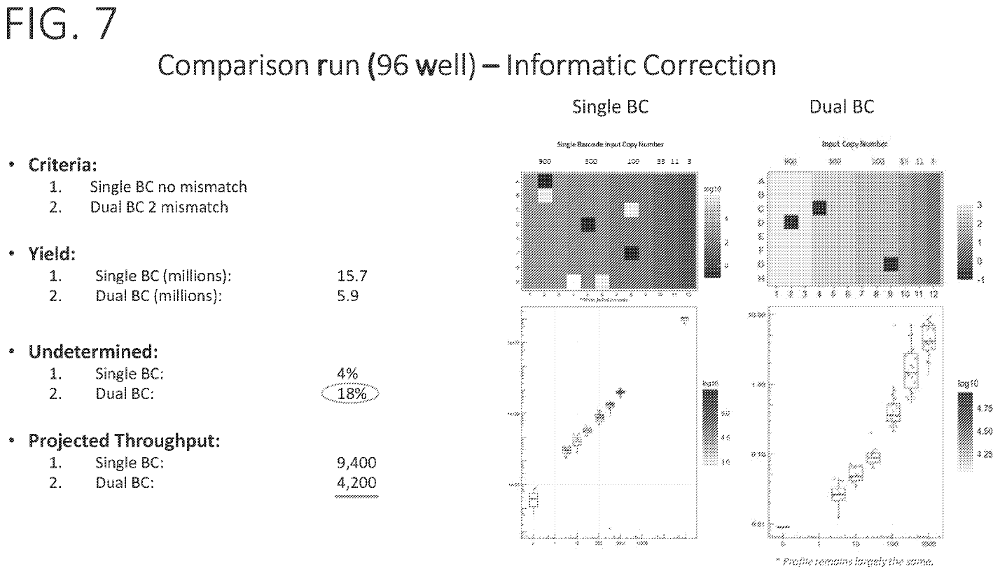

In some embodiments, a plurality of background signals are obtained, and by distinguishing the signal from sequencing the target at one location on the flow cell within the plurality of signals from sequencing the target at all the locations on the flow cell by the location of the signal from sequencing the SIR, the background signal and the signal from sequencing the target at that location can be distinguished. In some embodiments, the first read and the second read each generate their own background signals, and the background signals can be distinguished from the first read and second read signals from sequencing the target at one location on the flow cell by the location of the signals from sequencing the SIR. FIG. 7 depicts the heat maps from methods comprising a SIR or a dual barcode or index system, and the signal from sequencing the target by using the location of the signal from sequencing the SIR results in a much stronger signal (e.g., having a log 10 signal between 2 and 3) compared to results using traditional barcodes and distinguishing one signal from sequencing the target from the next by the sequence of the target (e.g., having a signal log 10 signal from 3 to 4).

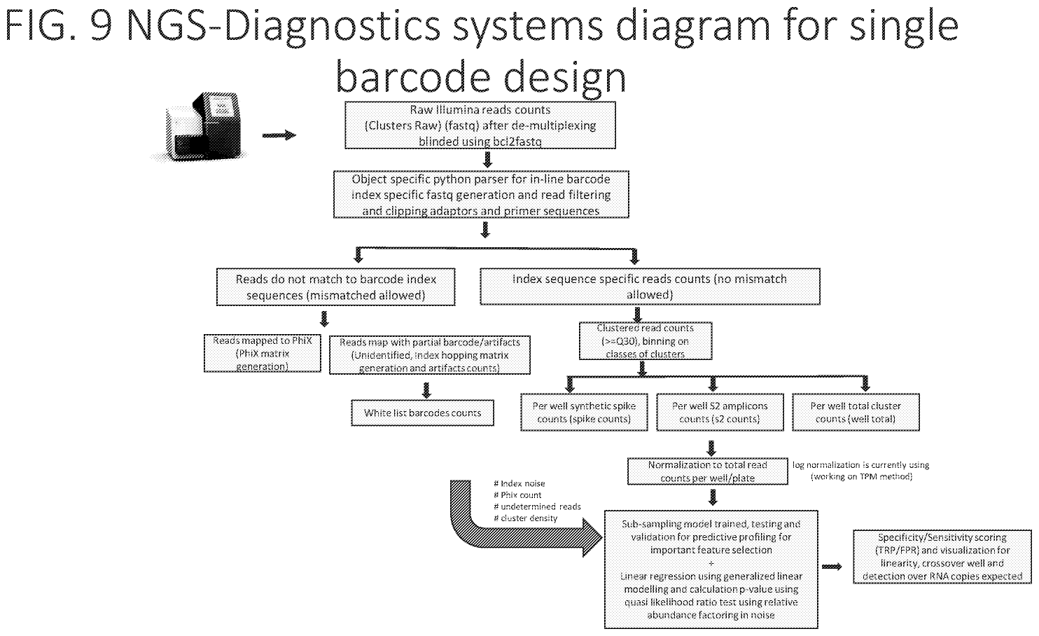

FIG. 9 depicts a step of using an object specific python parser to generate a SIR (i.e., barcode or index) specific fastq library file, and in addition to filter the reads and clip adapter and primer sequences from the read information. This object specific python parser is illustrative, and direct R, C++, or other methods of generating a fastq library file or another library file may be used. In some embodiments, no mismatches are allowed in the SIR sequencing reads. In some embodiments, 1, 2, 3, or 4, mismatches are allowed in the SIR sequencing reads. It is understood that more mismatches are allowed when the number of samples decreases and whether there is some redundancy built into the SIR to allow for mismatching.

In some embodiments, the method further comprises identifying the presence or absence of the first target in the sample. In some embodiments, the presence of the target in the sample is identified by the presence of at least one cluster having the signal from at least a first read sequencing of the target and a signal for the SIR identifying said sample in the cluster-differentiated-by-SIR library. In some embodiments, the absence of the target in the sample is identified by the absence of at least one cluster having the signal from at least the first read sequencing of the target or the signal for the SIR identifying said sample in the cluster-differentiated-by-SIR library. In some embodiments, the presence of the target in the sample is further identified by the one cluster also having a signal from at least the second read sequencing of the target. In some embodiments, the first read sequencing and second read sequencing of the target is compiled, and the presence of the target in the sample is identified by having at least one cluster having the sequence of the target and the signal for the SIR identifying said sample in the cluster differentiated library. In some embodiments, the first read sequencing and second read sequencing of the target is compiled, and the absence of the target in the sample is identified by having no clusters having the sequence of the target or the signal for the SIR identifying said sample in the cluster differentiated library.

In some embodiments, the method further comprises identifying whether a mutation is present in or absent in the target in the sample by comparing the sequence of the target when the sample is present to a reference sequence of the target or to the sequences of the target when present in the other samples. In some embodiments, the presence of the mutation occurs when the sequence of the first target in the sample differs in at least one, two, three, four, five, six, seven, eight, nine, ten, 20, 30, or 40 nucleotides from the reference sequence of the target or the sequences of the target in other samples. In some embodiments, the absence of the mutation occurs when the sequence of the target in the sample is identical to the reference sequence of the target and the sequences of the target in the other samples.

In some embodiments, two or more targets are identified in the sample. In some embodiments, the presence of the each of the two or more targets is identified by the above-noted methods. In some embodiments, the absence of the target is identified by the above-noted methods. In some embodiments, the method comprises from 1000 to 5000 samples, wherein the number of samples identified as having the target present over the number of samples having the target present is from 0.54 to 1.0. In some embodiments, the method comprises 10000 to 76000 samples. In some embodiments, the method comprises 100000 samples.

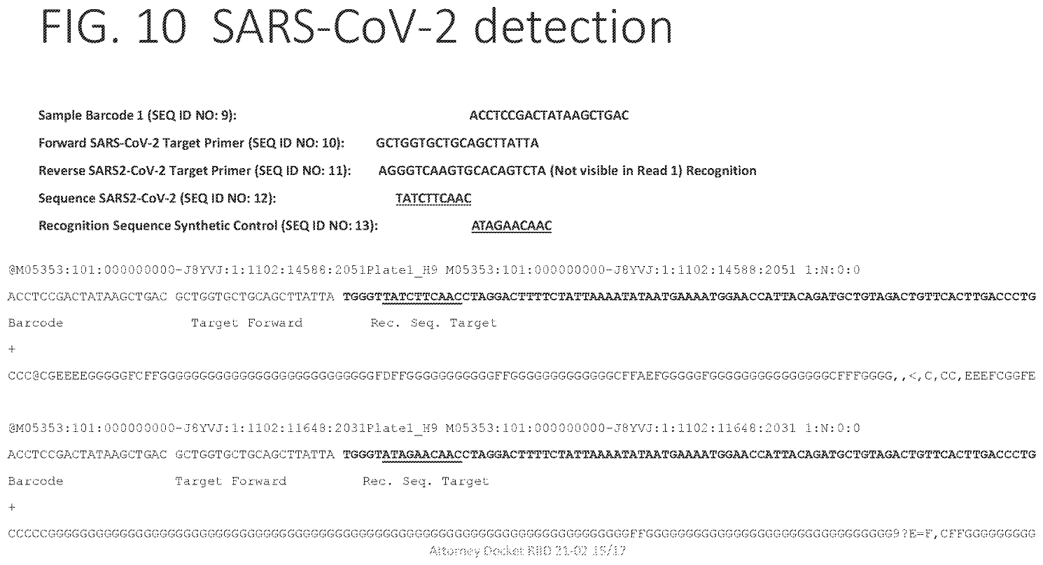

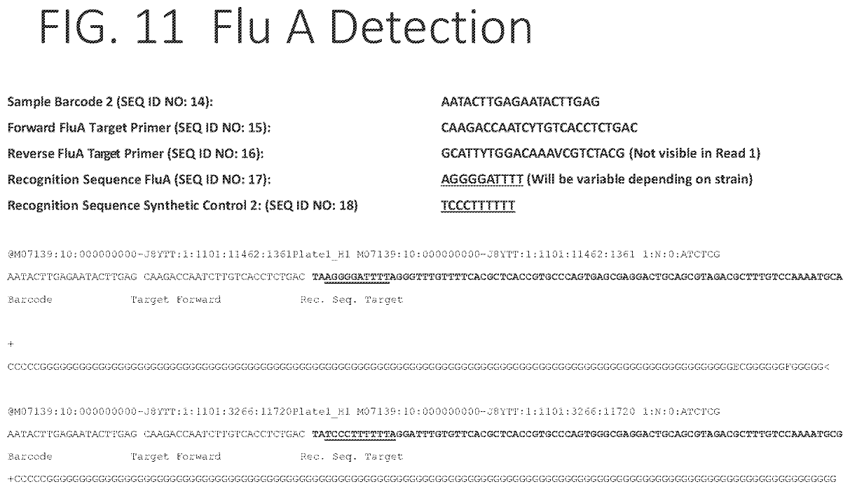

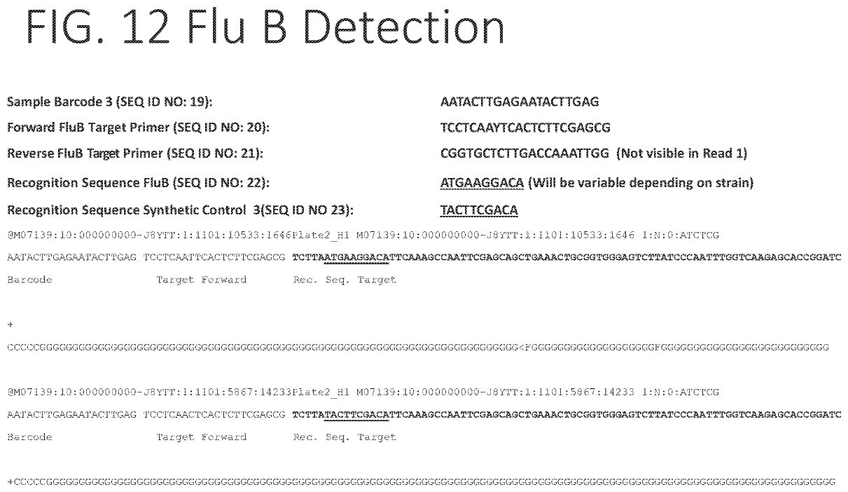

In some embodiments, to each sample, a synthetic target is added, the synthetic target comprising regions to which the first and second target primers can bind, and having an intermediary region that differs from the target. In some embodiments, the synthetic target is read in the first and/or second read. In some embodiments, the presence of the synthetic target is identified before the presence or absence of the target is identified. Thereby, the synthetic target is used as a control to ensure that the conditions for producing the single-stranded products, the clusters, and the first and/or second reads were appropriate for that sample. FIGS. 10-12 depict synthetic targets for SARS-COV-2 (FIG. 10), Influenza A (FIG. 11), and Influenza B (FIG. 12).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

FIG. 3A provides a preferred example wherein the first adapter and second adapter comprise first and second target primers, and the first and second adapters themselves function as primers for an amplification of the target. In this embodiment, the first and second primer binding regions each comprise single-stranded nucleic acid binding regions and sequencing primer binding regions. In this embodiment, the first sequencing primer, when elongated during the sequencing-by-synthesis, sequences the SIR, and in line and downstream, a first region in the target.

### Example 2

FIGS. 3B-3D provide for another preferred embodiment using adapters having the same structure as in Example 1, but wherein the target is first reverse transcribed using either random primers or the target primers (FIG. 3B), thereby producing a single-stranded cDNA. To this the first adapter, is annealed and elongated (FIG. 3C) thereby producing a product comprising the first primer binding region, the SIR, and the target. To this first product is annealed the second adapter, and the second adapter is elongated, thereby obtaining a second single-stranded product comprising the first primer binding region, the SIR, the target, and the second primer binding region (FIG. 3D).

### Example 3

FIGS. 3E and 3F provide for another preferred embodiment using target primers to amplify the target, and then ligating adapters. Optionally, the target is first reverse transcribed using either random primers (FIG. 3E). The target primers are used to amplify the target (FIGS. 3E and 3F). To this the first and second adapters are ligated thereby producing a second single-stranded product comprising the first primer binding region, the SIR, the target, and the second primer binding region. In this embodiment, a 5′ overhang is used to provide for the appropriate orientation of the adapters. Y adapters, as described above, may also be used. Overhangs or blunt ends may be used for the ligation.

### Example 4

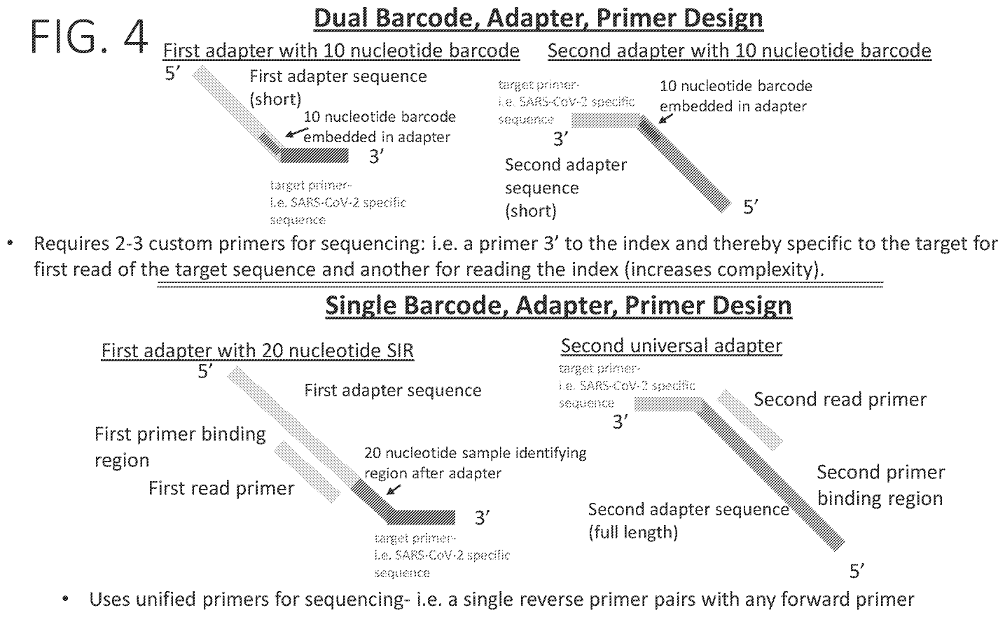

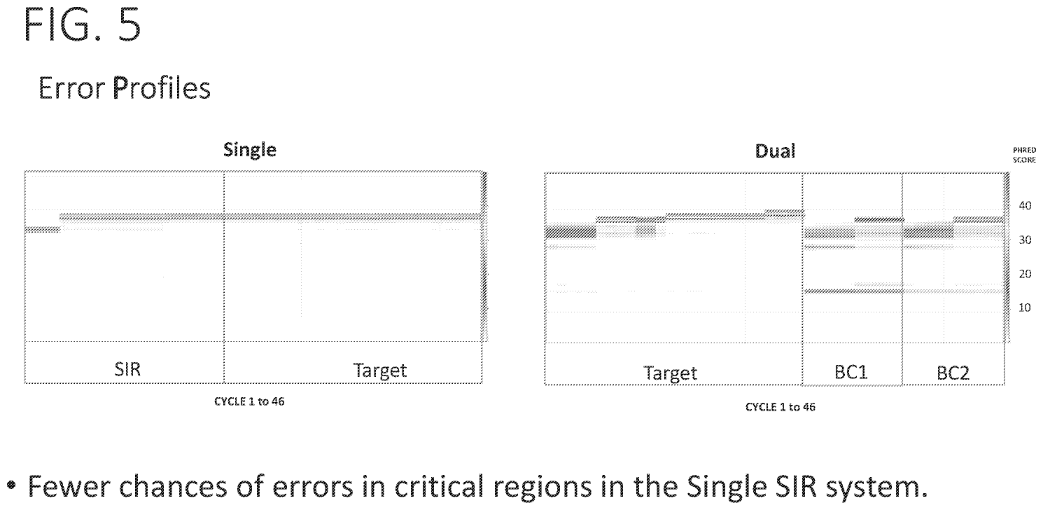

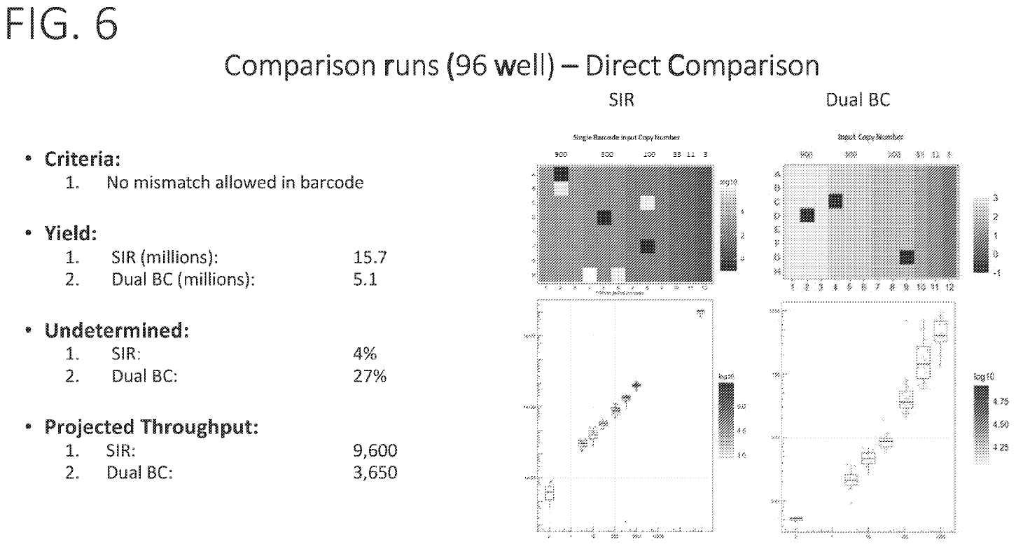

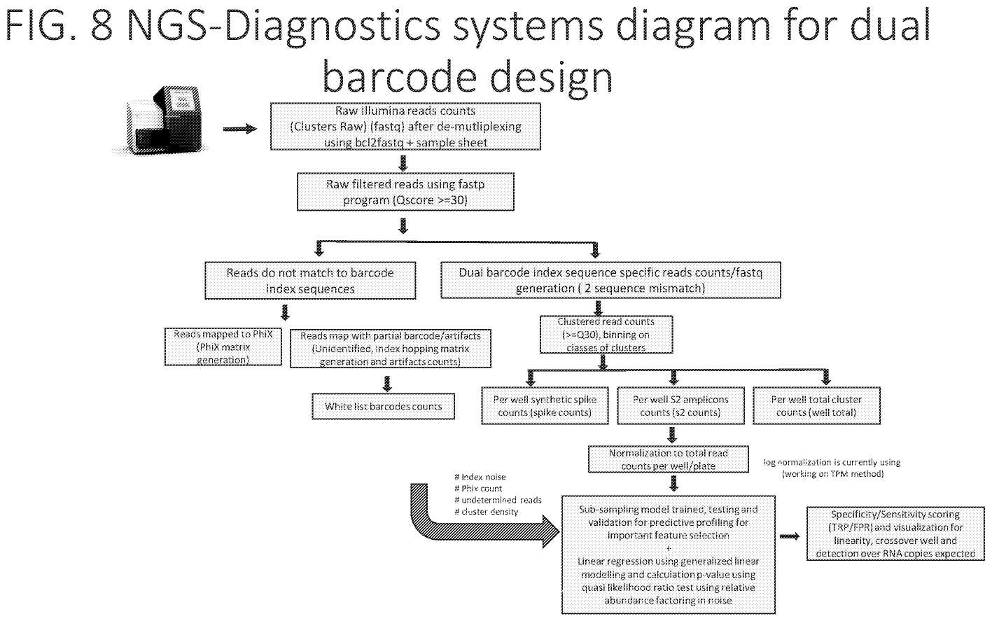

FIG. 4 provides a comparison of the adapters used in the following study to produce the error profiles in FIG. 5; the heatmaps, yields; error rates, projected throughputs, and standard curves with the informatics uncorrected and informatics corrected results depicted in FIGS. 6 and 7 respectively. In this experiment, 96 samples were run and the presence or absence of SARS-CoV-2 in these samples were detected using the methods depicted in FIGS. 8 and 9. FIG. 10 depicts the targeting and first read results for SARS-COV-2. The standard curves, heatmaps, yields, undetermined sample percentages, and projected throughput using the above-noted methods were unexpected compared to the same results using existing dual barcode methods, regardless of whether the results had informatics correction or not.

### Example 5

The same methods as in Example 5 were applied to a group of 1000 samples. Five out of 6 of the attempts using existing methods involving dual barcode designs and distinguishing clusters based on their respective sequences of the targets (i.e., Octant methods) failed to provide discernable data. The library of sequence data was not able to be resolved by the existing methods and the results from the entire run collapsed. On the sixth run, 69% of the samples using the existing methods were able to have SARS-COV-2 nucleic acid detected and another 31% of the samples known to have SARS-COV-2 nucleic acid failed to have the SARS-COV-2 nucleic acid detected. In contrast, using the above-noted methods, at least 93% of the samples that contained SARS-COV-2 nucleic acid were identified by the methods as containing SARS-CoV-2 nucleic acid. In contrast to the existing methods, the methods-described herein are unexpected. Projections were further performed to determine where the existing Octant methods would completely fail (even though they failed in 5 out of the 6 other runs). Based on the results from the 6th run, 1% of samples that have SARS-COV-2 nucleic acid would be identified as having SARS-COV-2 nucleic acid using the Octant methods when there are 5000 samples in the run.

### Example 6

We sought to determine the maximum sequencing surveillance output of the ubiquitous Illumina MiSeq instrument. We initially believed the capability to be around 5,000 unique samples. In a stepwise fashion we increased the number of samples we loaded into the machine 1000-2000 at a time until we reached the limit where the assay began to lose sensitivity as read count per sample dropped below the minimum threshold required to make accurate positive or negative calls. This limit was determined to be 12,578, yielding 11,995 reads passing quality control filters (4.6% fail rate) (Table 1).

TABLE 1

| | Passed | Failed | Average Depth | Average Depth |
|---|---|---|---|---|
| Platform | QC | QC | CTRL | Target |
| iSeq | 2,391 | 105 (4.2%) | 319 ± 39 | 1259 ± 431 |
| MiSeq | 11,995 | 583 (4.6%) | 244 ± 192 | 1,733 ± 1331 |
| NextSeq | 46,712 | 1,116 (2.3%) | 593 ± 521 | 4,331 ± 1649 |

### Example 7

In order to examine the multiplexing capability of the NGS Surveillance platform, a pooled library was generated containing previously validated sample pools from SARS-COV-2 (S-gene and E-gene), Influenza A (FluA), and Influenza B (FluB). Total number of successfully validated unique samples tested on a MiSeq for each target in the multiplexed run are 1,514 of 1,536 (98.6% pass rate) for SARS-COV-2, 1,508 of 1,536 for FluA (98.2% pass rate), and 1518 of 2,304 (65.9% pass rate) for FluB. Uniquely barcoded FluB primers showed reduced sequencing capability with the designed primers with only 65.9% passing quality control filters. This is mitigated in the multiplexing context by requiring far fewer functional probes than a single target assay (1,500). A single target FluB assay should include a reevaluation of the target site.

### Example 8

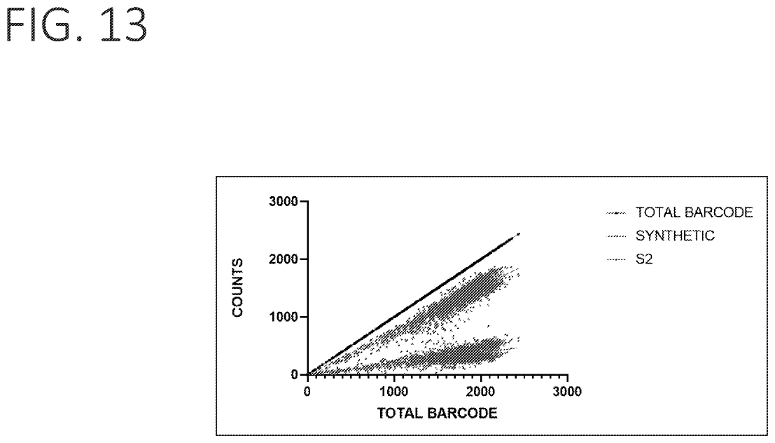

The Illumina iSeq 100 instrument, which has a read output of 4 million reads, is an excellent budget sequencer that is capable of being deployed into austere environments. The machine requires very little setup and preventative maintenance and can be operated with minimal training, making it an excellent option for COVID-19 screening in remote locations. We expected a maximum capacity of 500 samples was attainable based on theoretical output of expected read counts. With optimization were able to successfully push the system to attain 2,391 out of 2,496 unique samples passing Quality Control filters, a fail rate of 4.2% (FIG. 13). The average depth of reads for the synthetic control RNA was 319±39, with average target RNA depth at 1,259±431 (Table 1). The instrument was tested with both single and dual gene target systems (S-gene and E-gene). Average run time on the sequencer is approximately 8 hours with library preparation requiring only 10 minutes to perform.

### Example 9

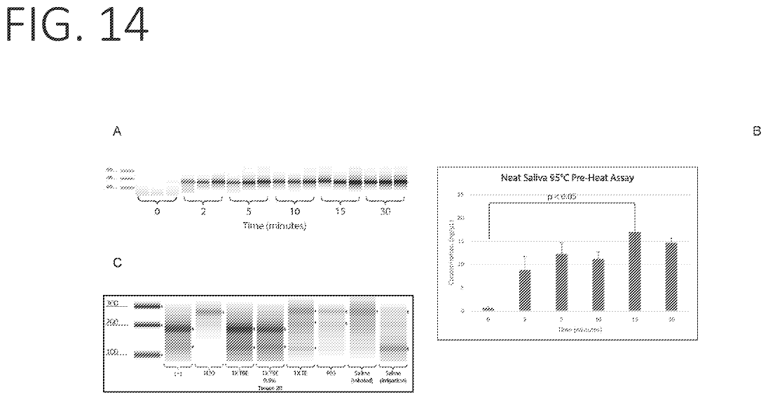

We sought to determine the best methodology for library preparation from neat saliva. Saliva contains enzymes such as proteases that are detrimental to RT-PCR amplification. We examined two methods to increase RT-PCR output; saliva sample pre-heating to inactivate disruptive enzymatic activity, and the addition of a diluent with or without detergents. For the pre-heat test, neat saliva was heated to 95 C in a thermocycler for between 0 and 30 minutes. RT-PCR was then performed using 2 μL of the heated neat saliva and concentration of amplicon cDNA was measured by TapeStation. Interestingly, at 2 minutes, an 8-fold increase in PCR product concentration was seen with maximum increase observed at 15 minutes (27-fold), p=0.041, student paired T-Test (FIG. 14A, B).

Diluting saliva can reduce viscosity and increase uniformity across samples. To test this, neat saliva was diluted 1:1 with the following reagents: molecular grade water, Tris-Borate-EDTA (TBE) with or without Tween 20, Tris-EDTA (TE), Phosphate Buffered Saline (PBS), 0.9% Saline solution (inhaled, and irrigation forms), or no dilution. Then, RT-PCR was performed using 2 μL each of the pre-diluted samples, and concentration of amplicon cDNA was measured by TapeStation. Our results indicate that water, 1×TE, PBS, and inhaled Saline can act as effective diluents for the increase of reliability of neat saliva in the NGS Surveillance Assay (FIG. 14C).

### Example 10

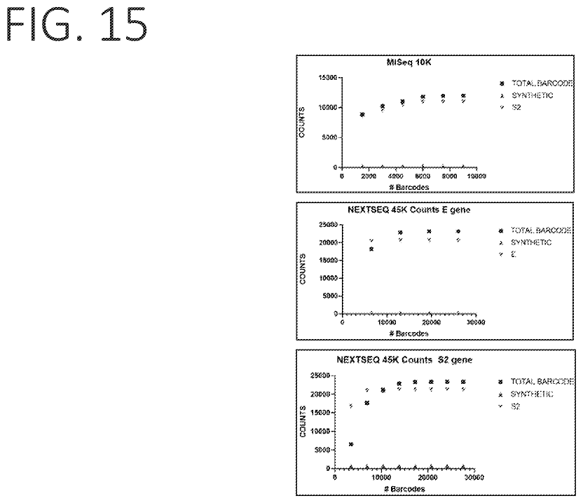

The barcode optimization was performed using two Illumina sequencing platforms, MiSeq V2.6 and NextSeq 2000. We tested 12,576 unique barcodes with 1000 RNA S2 gene copies with 500 synthetic copies in single MiSeq run. The run generated 29.5 million reads with cluster density of 2,114±61, 81.21% passing filter with Phix 13.55±0.63. The run total barcode depth for total barcode read depth of 1,733.392 with SPIKE count depth 244.0497774 and S2 gene depth of 1733.392 with 1.27 average mean absolute deviation. FIG. 15 shows the cumulative counts with seven sets of optimized barcodes with total number of reads in each set of barcodes. The 9000 barcodes on average had 90% showing expected read depth to call a positive signal accurately. Similarly, the two gene targets of SARS-COV-2, S2 gene with 24,097 barcodes and E gene for with 24,001 barcodes were optimized in single run on NextSeq 2000 with 419 million read output with 85% passing filters with PhiX of 14.9. The total barcodes passing the accuracy call for the E gene were 23,468 and 23,244 for S2.

### Example 11

The Illumina NextSeq 2000 is a mid-throughput sequencer capable of 200 million reads with a very simple library preparation methodology. The high number of reads makes the NextSeq the ideal instrument for surveillance when patient samples are in the tens of thousands. In order to test the capacity of the machine we sequenced amplicon pools made with both the S- and E-gene primers, examining a total of 47,828 unique samples. Of these, 46,712 passed quality control filters, a 2.3% fail rate, with an average depth of control synthetic reads at 593±521, and an average depth of target RNA reads at 4,331±1,649 (Table 1).

### Example 12

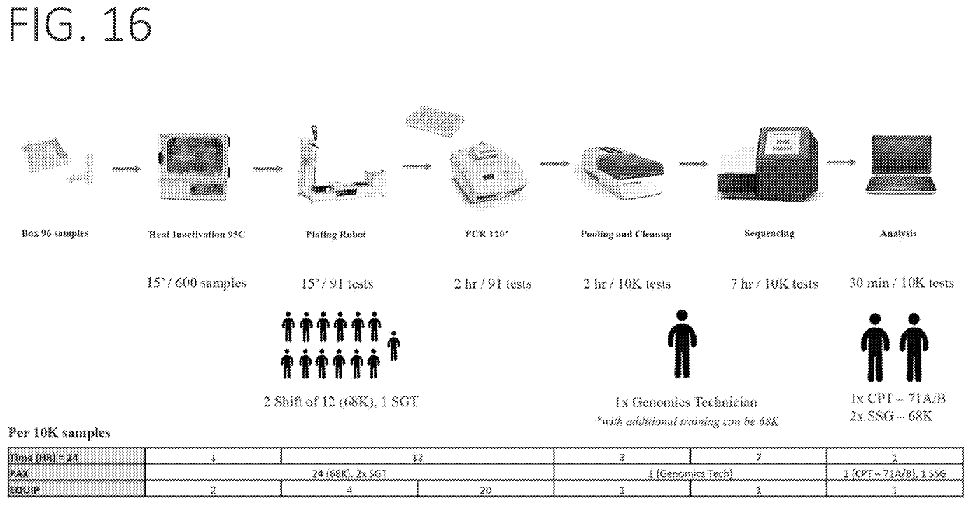

The primary goal of this project was to implement the methodology for massively pooled surveillance using an NGS reporter and document the process in terms of equipment, supplies, training & personnel, required footprint and develop standard operating procedures. Utilizing the lessons learned and materials supporting the methodology described previously we propose the following equipment and manpower footprint (FIG. 16) as the optimally required for a 10K samples processed every 24 hr hours. These samples would need to be in the neat saliva format. Approximately half as many nasopharyngeal samples can be processed utilizing the same footprint. The sample handling is laborious and increases collection time, shipping/packaging costs and biological waste.

To field test this capability, patient samples were sought and obtained from the United States Naval Academy. The samples were able to be surveilled using the NGS massive pooling assay now named Biodefence Mass Sequencing & Surveillance (BMASS). Enforcement discretion was provided allowing for the samples to be retested by a clinical diagnostic for NP swabs or to be resampled and tested by clinical diagnostic for NP swabs or saliva.

Due to the low per sample costs of the assay the ideal target population for BMASS are asymptomatic. To date resource limitations have restricted the preponderance of diagnostic testing to symptomatic presentations. Unfortunately, asymptomatic presentations create super spreaders in the absence of draconian enforcement of physical controls like masks, physical distancing, and other public health measures. Thus this assay represents an affordable paradigm shift in pooled screening of asymptomatic populations. Approximately 3,000 samples were collected from asymptomatic cohorts over the course of the project (Table 2). We observed a 0.34% positivity over all samples and to the best of our ability to ascertain there were no false negatives or false positives during the utilization of the assay. There was a 0.73% repeat rate which was below the 2% limit originally anticipated for the assay due to failed internal amplification controls (invalid) or discrepancies between replicates (repeat). Either condition triggers an automatic retesting of the sample. The saliva results shown requiring repetition (13.8%) are prior to optimization of the protocol.

TABLE 2
USNA patient sample surveillance totals

| Project Totals | NP SWAB | SALIVA v1 |
|---|---|---|
| Samples Tested | 2,455 | 500 |
| [symbol] Positivity | 10 (0.4%) | 3 (0.6%) |
| [symbol] False Negatives | 0 (0.0%) | 0 (0.0%) |
| [symbol] False Positives | 0 (0.0%) | 0 (0.0%) |
| [symbol] REP—Captures POS | 4 (0.2%) | 1 (0.2%) |
| [symbol] REP—True NEG | 14 (0.6%) | 12 (2.4%) |
| [symbol] REP—Invalid | 0 (0.0%) | 56 (11.2%) |
| Samples Tested | 2,455 | 500 |
| Positivity | 10 (0.4%) | 3 (0.6%) |
| False Negatives | 0 (0.0%) | 0 (0.0%) |
| False Positives | 0 (0.0%) | 0 (0.0%) |
| REP—Captures POS | 4 (0.2%) | 1 (0.2%) |
| REP True NEG | 14 (0.6%) | 12 (2.4%) |
| REP—Invalid | 0 (0.0%) | 56 (11.2%) |

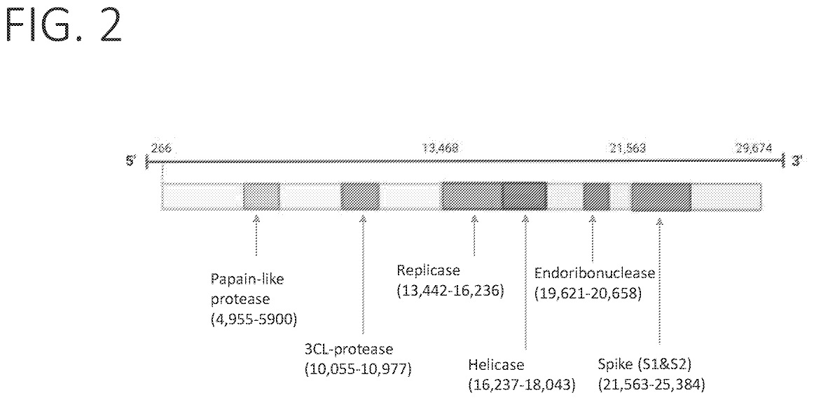

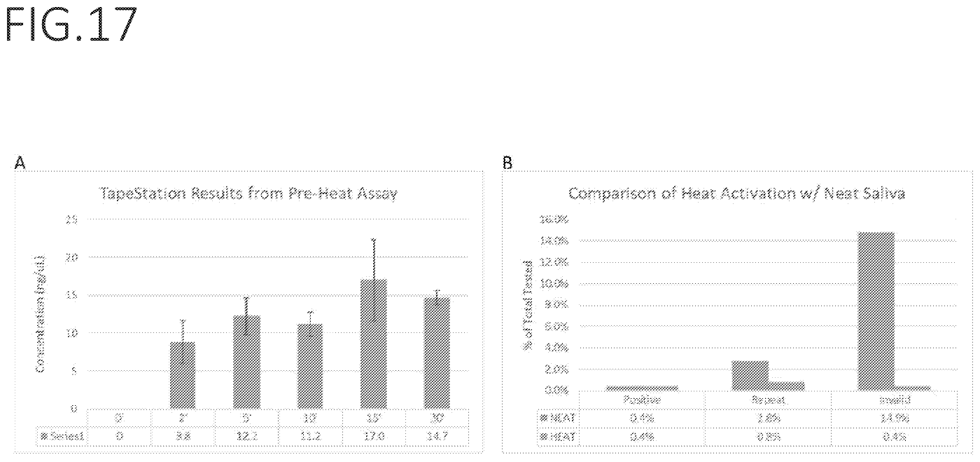

The ideal protocol optimization to reduce issues interfering with the amplification of the SC2 target was to heat inactivate. In brief, negative saliva samples failing the internal PCR control for amplification were spiked with 1000 copies of virus and subjected to the addition of a pre-heat step to denature enzymes thought to be active in neat saliva that would not have been similarly active in the swab sample due to the presence of inactivators and stabilizers found in various viral transport mediums (FIG. 17A). After heat inactivation the results indicated the samples requiring repetition ‘Repeat and Invalid’ went from 17.7% to 1.2% which brought the protocol in line with acceptable repetition rates (FIG. 17B). The n of population in FIG. 2B is 250 samples as opposed to the project summary 500 (Table 2), explaining the different repeat percentages. Pre-heating did not abrogate the ability to detect the known positive in this cohort. This work allowed for switching BMASS' ideal sample matrix submission to saliva. Large groups/formations can expedite sampling by providing prelabeled collection tubes explaining once and turning in the tube. Once the process was understood large savings in collection times and a less invasive collection method was achieved.

As a proof of concept for the applied value of this capability in the week of 24 Feb. 2021 the midshipmen of USNA surged over 100 symptomatic cases. This was ascribed to liberty in the community the previous weekend. Due to the introduction of the ALPHA variant a local surge in positivity (4.28%, Anne Arundel County, 25 Feb. 2021) was observed during that time period (https://coronavirus.maryland.gov/, accessed 12 Oct. 2021) and peaked 8 Apr. 2021 at 7.47%. That same week we were surveilling an asymptomatic cohort at USNA of 506 samples. Of these 9 samples were determined to be positive by the NGS surveillance assay and recommended for retesting by clinical diagnostic (Table 3). The surveillance samples in Table 3 were also tested by an RUO PCR assay to provide comparative CTs. None of these results were utilized for patient care or provided back to the patient. Of the patients identified, 1 was presymptomatic and had been admitted for care by the time the results were reported (48 hours post sampling) and 8 were asymptomatic and continued to present no disease throughout the course of infection. Under a different effort these samples were sequenced for whole genome characterization and were determined to be primarily (7 of 9) as ALPHA variant 7 days post NGS surveillance.

TABLE 3
USNA Asymptomatic Cohort

| | | | | NGS | | |
|---|---|---|---|---|---|---|
| PATIENT | | PCR | | | CTRL | TARGET |
| ID | SYMPTOMS | CTRL | TARGET | n= | DEPTH | DEPTH |
| NEG | — | 32.2 | — | 46 | 29886 ± 8612 | 217 ± 348 |
| POS | — | 32.1 | 32.7 | 12 | 24595 ± 6660 | 8786 ± 2488 |
| S001 | ASYMPTOMATIC | 31.8 | 17.6 | 1 | 440 | 37507 |
| S002 | ASYMPTOMATIC | 31.8 | 18.8 | 1 | 536 | 68965 |
| S003 | PRE- | 32.1 | 14.5 | 1 | 199 | 80833 |
| | SYMPTOMATIC | | | | | |
| S004 | ASYMPTOMATIC | 31.3 | 19.9 | 1 | 461 | 46253 |
| S005 | ASYMPTOMATIC | 31.7 | 17.5 | 1 | 1102 | 57004 |
| S006 | ASYMPTOMATIC | 31.5 | 18.5 | 1 | 883 | 62677 |
| S007 | ASYMPTOMATIC | 31.9 | 18.1 | 1 | 268 | 74210 |
| S008 | ASYMPTOMATIC | 32.1 | 24.3 | 1 | 3428 | 47710 |
| S009 | ASYMTPMATIC | 31.4 | 19.9 | 1 | 3206 | 48982 |

| | | | | | |
|---|---|---|---|---|---|
| S010-506 | — | NOT RUN | 497 | 26641 ± 10844 | 188 ± 324 |

To demonstrate the ability of the team to perform 10K samples in 1 week it was necessary to utilize stock virus at 1,000 copy dilutions. A sample set of equivalent size was not obtained via patient samples. In support of project 2157160950B the Operationalization team processed ˜46K samples. Dependent on probe deliveries at multiple points it was necessary to process >10K in one week (FIG. 15). In this example run 12,576 unique barcodes were run with stock virus. This resulted in 11,995 samples passing QC filters for detection and set the capacity limit for the MiSeq which is the instrument of choice for the method operationalized in this project.

### Example 14

For workflow primer and barcode generation first, all possible combination of barcodes are generated using DNA-Barcodes python package. (https://www.bioconductor.org/packages/release/bioc/html/DNABarcodes.html). The program uses following conditions for minimum Levenshtein pairwise distance (3), length (20), minimum (20%) and maximum GC (90%) content, filtering homopolymers. It also excludes specific patterns in the barcode sequences GTTCIATTC. It typically generates 300K barcodes. It also looks for a minimum number of different bases between barcodes.

This initial barcode set is further processed for testing the specificity to forward/reverse primers for target gene and sequencing primers. This is a customized pipeline build using python package primer3, Levenshtein and DPA initial primer/barcode design package.

It has five step filtering processes, described below:
1. In this filtering step, all barcodes are scanned across for minimum Levenshtein distance using Conways Lexicode algorithm for hamming distances codes. Where all the barcodes are binned to look minimum 3 hamming distance compared to each barcodes.
2. In this step algorithm, it calculates GC content using following formula:

$$GC = Count ( G + C ) / Count ( A + T + G + C ) * 100 \%$$

Where GC filters are applied here with a Max GC of ≥75% and a Min GC of ≤35%
3. This step has multiple steps which include the calculation of hairpin ΔG and Tₘ and calculation of Homodimer.
   Calculation of the hairpin ΔG:
   The calculation of the hairpin ΔG and Tₘ uses all four primers (R/F sequencing primer and R/F target gene specific primers) against the barcode sequence. This uses the calcHairpin function from the python package primer3 (https://pypi.org/project/primer3-py/) that calculates hairpin formations thermodynamic for given sequences and saves the value for ΔG and Tₘ from the object along with primer pair sequence along with barcodes. It calculates the cutoffs as per with specific primer barcode pair orientations. These cutoffs are used to filter down the barcodes.
   Calculation of HomoDimer:
   Hairpin and self-dimer Tₘ and ΔG are calculated using primer3.py. Then random test is performed on barcode combinations with sample depth of 50000.
4. Well specific heterodimers calculations calculate all the possible combinations of possible primer pairing within one well. The ΔG is calculated and the minimum value is reported.
   Barcodes are then filtered based on the following parameters:
      Per barcode number of target, synthetic read counts >500
      Total read count counts per barcode should >2000
      The passing threshold should be >20% (Target read count+Synthetic read count/total read count)
      Distance of barcode to primer and target sequence is set during read filtering.

### REFERENCES

The disclosures of each and every patent, patent application, and publication cited herein are hereby incorporated herein by reference in their entirety. While this invention has been disclosed with reference to specific embodiments, it is apparent that other embodiments and variations of this invention may be devised by others ordinarily skilled in the art without departing from the true spirit and scope of the invention. The appended claims are intended to be construed to include all such embodiments and equivalent variations.

What is claimed is:

1. A method of detecting in vitro the presence or absence of a first target in a sample, the first target being a single strand of nucleic acid, the method comprising:
i) For two or more samples, admixing with each sample:
A) a first reaction mixture comprising a first adapter and a second adapter, the first adapter comprising from 5′ to 3′ a first primer binding region, a first sample identifying region (SIR), and a first target primer, each first SIR comprising a sequence that identifies each sample, the second adapter comprising from 5′ to 3′ a second primer binding region and a second target primer, the admixing being under conditions when the first target is present in the sample wherein:
I) one of the first adapter or the second adapter anneals to the first target, wherein a one of the first target primer or the second target primer anneals to the first target, thereby obtaining a first target-bound adapter;
II) the first target-bound adapter is elongated to obtain a first single-stranded product comprising: I) the first target and IIa) the first primer binding region and first SIR or IIb) the second primer binding region;
III) the other of the first adapter or the second adapter anneals to the first single-stranded product, wherein the other of the first target primer or the second target primer anneals to the first target, thereby obtaining a second target-bound adapter; and
IV) the second target-bound adapter is elongated, thereby obtaining a second single-stranded product comprising the first primer binding region, first SIR, the first target, and the second primer binding region; or
B) a second reaction mixture comprising the first target primer and the second target primer, the admixing being under conditions when the first target is present in the sample wherein:
I) the first target is amplified, thereby obtaining a double-stranded first target; and
II) further admixing a first polynucleotide and a second polynucleotide to the second reaction mixture, the first polynucleotide comprising the first primer binding region and the first SIR, the second polynucleotide comprising the second primer binding region, wherein the first polynucleotide and second polynucleotide are ligated to the double-stranded target, the first polynucleotide being ligated to an opposite end of the double-stranded target as the second polynucleotide, the first SIR being proximal and the first primer binding region being distal to the target; thereby obtaining at least the second single-stranded product;
thereby obtaining a first modified sample from each admixing of each of the two or more samples;
ii) pooling two or more of the first modified samples, thereby obtaining a pooled sample;
iii) flowing the pooled sample over a flow cell comprising a substrate, a first single-stranded nucleic acid, and a second single-stranded nucleic acid, the 5′ end of each of the first single-stranded nucleic acid and the second single-stranded nucleic acid being bound to the substrate, the first single-stranded nucleic acid being capable of a first annealing to the first primer binding region, and the second single-stranded nucleic acid being capable of a second annealing to the second primer binding region, the flowing being under conditions that permit at least one of the first annealing or the second annealing;
iv) bridge-amplifying the second single-stranded product thereby obtaining two or more clusters, each cluster comprising a second primer binding region-bound second single-stranded product and having a location on the flow cell, the second primer binding region-bound second single-stranded product comprising the second single-stranded product wherein the 5′ end of the second primer binding region is bound to the substrate, each second primer binding region-bound second single-stranded product in each cluster having the same SIR and thereby being from the same sample;
v) annealing a first sequencing primer to the first primer binding region of the second primer binding region-bound second single-stranded product;
vi) sequencing-by-synthesis the first SIR and at least a first sequence within the first target, the first sequence within the first target being proximal to the first SIR, thereby obtaining a plurality of first read signals comprising a plurality of first signals and a plurality of second signals, each of the first signals and each of the second signals having a location on the flow cell, the first signal comprising the signal from sequencing the first SIR, and the second signal comprising the signal from sequencing the at least the first sequence within the first target;
vii) generating a cluster-differentiated-by-SIR library by:
A) identifying the location of each cluster on the flow cell by the location of each first signal on the flow cell; and
B) distinguishing within the plurality of second signals, each second signal by the location of each first signal on the flow cell, thereby distinguishing the second signal for one cluster from the second signals for all other clusters; and
viii) identifying the presence or absence of the first target in the sample, the presence of the first target in the sample being identified by the presence of at least one cluster having the second signal and the first signal for the first SIR identifying said sample in the cluster-differentiated-by-SIR library, and the absence of the first target in the sample being identified by the absence of one cluster having the second signal and the first signal for the first SIR identifying said sample in the cluster-differentiated-by-SIR library.

2. The method of claim 1, the first read signals further comprising a plurality of first background signals, each first background signal being from a location on the flow cell not having clusters, and in vii) B) distinguishing each of the first background signals from the location of each first signal on the flow cell, thereby identifying whether the second signal for one cluster is distinguishable from the first background signal.

3. The method of claim 1, further comprising:
after vi)
a. annealing the first primer binding region of the second primer binding region-bound single-stranded product to the first single-stranded nucleic acid and elongating the first single-stranded nucleic acid, thereby obtaining a first primer binding region-bound second single-stranded product comprising the second single-stranded product wherein the 5′ end of the first primer binding region is bound to the substrate;
b. annealing a second sequencing primer to the second primer binding region of the first primer binding region-bound second single-stranded product; and
c. sequencing-by-synthesis at least a second sequence within the first target, the second sequence within the first target being proximal to the second primer binding region, thereby obtaining a plurality of second read signals comprising a plurality of third signals, each of the third signals having a location on the flow cell, the third signal comprising the signals from sequencing the at least the second sequence within the first target, the first sequence within the first target together with the second sequence within the first target comprising the first target; and
in vii) generating the cluster-differentiated-by-SIR library further by:
C) distinguishing within the plurality of the third signals, each third signal by the location of each first signal on the flow cell, thereby distinguishing the third signal for one cluster from the third signals for all other clusters.

4. The method of claim 3, the second read signals further comprising a plurality of second background signals, each of the second background signals having a location on the flow cell not having clusters, and in vii) C) distinguishing each of the second background signals from the location of each first signal on the flow cell, thereby identifying whether the third signal for one cluster is distinguishable from the second background signal.

5. The method of claim 3, wherein in viii), the presence of the first target in the sample is further identified by the presence of at least one cluster having the third signal, the second signal, and the first signal for the first SIR identifying said sample in the cluster-differentiated-by-SIR library, and the absence of the first target in the sample is further identified by the absence of one cluster having the third signal, the second signal, and the first signal for the first SIR identifying said sample in the cluster-differentiated-by-SIR library.

6. The method of claim 3, further comprising compiling the second sequence within the target and the first sequence within the target to generate the sequence of the first target when present in the sample.

7. The method of claim 6, further comprising identifying whether a mutation is present or absent in the first target in the sample by comparing the sequence of the first target when present in the sample to a reference sequence of the first target or the sequences of the first target when present in other samples, the presence of the mutation occurring when the sequence of the first target in the sample differs in at least one nucleotide from the reference sequence of the first target or the sequences of the first target in other samples, the absence of the mutation occurring when the sequence of the first target in the sample is identical to the reference sequence of the first target and the sequences of the first target in the other samples.

8. The method of claim 6, wherein in viii) the presence of the first target in the sample is identified by the presence of at least one cluster having the sequence of the first target and the first signal for the first SIR identifying said sample in the cluster-differentiated-by-SIR library, and the absence of the first target in the sample is identified by the absence of one cluster having the sequence of the first target and the first signal for the first SIR identifying said sample in the cluster-differentiated-by-SIR library.

9. The method of claim 1 further comprising detecting in vitro the presence or absence of a second target in the sample, the second target being a single strand of nucleic acid, the method further comprising:
before ii) admixing with each sample:
C) a third reaction mixture comprising a third adapter and a fourth adapter, the third adapter comprising from 5′ to 3′ the first primer binding region, a second SIR, and a third target primer, each second SIR comprising a sequence that identifies each sample, the fourth adapter comprising from 5′ to 3′ the second primer binding region and a fourth target primer, the admixing being under conditions when the second target is present in the sample wherein:
I) one of the third adapter or the fourth adapter anneals to the second target, wherein a one of the third target primer or the fourth target primer anneals to the second target, thereby obtaining a third target-bound adapter;
II) the third target-bound adapter is elongated to obtain a third single-stranded product comprising: a) the second target and b1) the first primer binding region and the second SIR or b2) the second primer binding region;
III) the other of the third adapter or the fourth adapter anneals to the third single-stranded product, wherein the other of the third target primer or the fourth target primer anneals to the second target, thereby obtaining a fourth target-bound adapter; and
IV) the fourth target-bound adapter is elongated, thereby obtaining a fourth single-stranded product comprising the first primer binding region, the second SIR, the second target, and the second primer binding region; or
D) a fourth reaction mixture comprising the third target primer and the fourth target primer, the admixing being under conditions when the second target is present in the sample wherein:
I) the second target is amplified, thereby obtaining a double-stranded second target; and
II) further admixing a third polynucleotide and the second polynucleotide to the second reaction mixture, the third polynucleotide comprising the first primer binding region and the second SIR, wherein the third polynucleotide and second polynucleotide are ligated to the double-stranded target, the third polynucleotide being ligated to an opposite end of the double-stranded target as the second polynucleotide, the second SIR being proximal and the first primer binding region being distal to the target; thereby obtaining at least the fourth single-stranded product;
thereby obtaining a second modified sample from admixing each sample with the second reaction mixture;
in ii) pooling the second modified samples and the first modified samples, thereby obtaining the pooled sample;
in iv) further bridge-amplifying the fourth single-stranded product, each cluster comprising the second primer binding region-bound second single-stranded product or a second primer binding region-bound fourth single-stranded product, the second primer binding region-bound fourth single-stranded product comprising the fourth single-stranded product wherein the 5′ end of the second primer binding region is bound to the substrate, each second primer binding region-bound fourth single-stranded product in each cluster having the same SIR and thereby being from the same sample;
in v) annealing the first sequencing primer to the first primer binding region of the second primer binding region-bound fourth single-stranded product;
in vi) further sequencing-by-synthesis the second SIR and at least a third sequence within the second target, the third sequence within the second target being proximal to the second SIR, wherein the plurality of first read signals further comprises a plurality of fourth signals and a plurality of fifth signals, each of the fourth signals and each of the fifth signals having a location on the flow cell, the fourth signal comprising the signal from sequencing the second SIR, and the fifth signal comprising the signal from sequencing the at least the third sequence within the second target;
in vii) generating the cluster-differentiated-by-SIR library further by: in A) identifying the location of each cluster by the location of each first signal or each fourth signal on the flow cell; and C) distinguishing within the plurality of fifth signals, each fifth signal by the location of each fourth signal on the flow cell, thereby distinguishing the fifth signal for one cluster from the fifth signals for all other clusters; and
ix) identifying the presence or absence of the second target in the sample, the presence of the second target in the sample being identified by the presence of at least one cluster having the fifth signal and the fourth signal for the second SIR identifying said sample in the cluster-differentiated-by-SIR library, and the absence of the second target in the sample being identified by the absence of one cluster having the fifth signal and the fourth signal for the second SIR identifying said sample in the cluster-differentiated-by-SIR library.

10. The method of claim 9, further comprising:
after vi):
a. annealing the first primer binding region of the second primer binding region-bound second single-stranded product to the first single-stranded nucleic acid and elongating the first single-stranded nucleic acid, thereby obtaining a first primer binding region-bound second single-stranded product comprising the second single-stranded product wherein the 5′ end of the first adapter is bound to the substrate and annealing the first primer binding region of the second primer binding region-bound fourth single-stranded to the first single-stranded nucleic acid and elongating the first single-stranded nucleic acid, thereby obtaining a first primer binding region-bound fourth single-stranded product comprising the fourth single-stranded product wherein the 5′ end of the first primer binding region is bound to the substrate;
b. annealing a second sequencing primer to: the second primer binding region of the first primer binding region-bound fourth single-stranded product and the second primer binding region of the first primer binding region-bound second single-stranded product; and
c. sequencing-by-synthesis at least a second sequence within the first target and at least a fourth sequence within the second target, the second sequence within the first target being proximal to the second primer binding region, the fourth sequence within the second target being proximal to the second primer binding region, thereby obtaining a plurality of second read signals comprising a plurality of third signals and a plurality of sixth signals, each of the third signals and sixth signals having a location on the flow cell, the third signal comprising the signals from sequencing the at least the second sequence within the first target, the first sequence within the first target together with the second sequence within the first target comprising the first target, the sixth signals comprising the signals from sequencing the at least the fourth sequence within the second target, the fourth sequence within the second target together with the third sequence within the second target comprising the second target; and
in vii) generating the cluster-differentiated-by-SIR library further by:
C) distinguishing within the plurality of the third signals, each third signal by the location of each first signal on the flow cell, thereby distinguishing the third signal for one cluster from the third signals for all other clusters; and
D) distinguishing within the plurality of the sixth signals, each sixth signal by the location of each fourth signal on the flow cell, thereby distinguishing the sixth signal for one cluster from the sixth signals for all other clusters.

11. The method of claim 9, wherein for each sample, the first SIR and second SIR have the same sequence.

12. The method of claim 1, wherein the first SIR comprises 15 or more, or 20 or more nucleotides.

13. (canceled)

14. (canceled)

15. The method of claim 1, wherein the target comprises DNA or wherein the target comprises RNA, and in i) A) II), i) A) IV), or i) B) I), the elongating comprises reverse transcription.

16. The method of claim 1, wherein the first target is from: sudden acute respiratory syndrome-associated coronavirus (SARS-COV), SARS-COV-2, or an influenza virus, or wherein the first target is SARS-COV-2 and the second target is an influenza virus.

17. (canceled)

18. (canceled)

19. The method of claim 1, comprising from 1000 to 5000 samples, wherein the number of samples identified as having the target present over the number of samples having the target present is from 0.54 to 1.0.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A kit for collecting and processing samples for detecting the presence or absence of one or more targets in said samples, said kit comprising
i. a master mix, comprising a reaction buffer, a polymerase, and dNTPs,
ii. For each target, a first adapter and a second adapter, the first adapter comprising from 5′ to 3′ a first primer binding region, a sample identifying region (SIR), and a first target primer, and the second adapter comprising from 5′ to 3′ a second primer binding region and a second target primer,
iii. and a positive control specific for each target.

45. The kit of claim 44, wherein the first primer binding region comprises SEQ ID NO. 5.

46. The kit of claim 44, wherein the second primer binding region comprises SEQ ID NO. 6

47. (canceled)

48. (canceled)

49. The kit of claim 44, wherein the first target primer comprises SEQ ID NO. 7, SEQ ID NO 10, SEQ ID NO: 15, SEQ ID NO: 20, or SEQ ID NO. 25.

50. The kit of claim 44, wherein the second target primer comprises SEQ ID NO. 8, SEQ ID NO 11, SEQ ID NO: 16, SEQ ID NO: 21, or SEQ ID NO. 26.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)
